(19) United States
(12) Patent Application Publication
(10) Pub. No.: US 2010/0209941 A1
(43) Pub. Date: Aug. 19, 2010

(54) METHOD, DEVICE AND KIT FOR DETERMINING CONDITIONS RELATED TO A DYSFUNCTION OF THE RENAL PROXIMAL TUBULE

(75) Inventors: François Jouret, Wezembeek-Oppem (BE); Philippe Gailly, Bruxelles (BE); Olivier Devuyst, Bruxelles (BE)

Correspondence Address: KNOBBE MARTENS OLSON & BEAR LLP, 2040 MAIN STREET, FOURTEENTH FLOOR, IRVINE, CA, 92614, US

(73) Assignee: Universite Catholique de Louvain, Louvain-la-Neuve (BE)

(21) Appl. No.: 12/524,993

(22) PCT Filed: Feb. 12, 2008
(86) PCT No.: PCT/EP08/51690

§ 371 Date: Jul. 29, 2009

(30) Foreign Application Priority Data
Feb. 12, 2007 (EP) 07447011.3

(51) Int. Cl. G01N 33/573; C12Q 1/00; C12M 1/34

(52) U.S. Cl. 435/7.4; 435/4; 435/287.2

(57) ABSTRACT

A method, device and kit are disclosed for determining a condition related to dysfunction of the Renal Proximal Tubule (RPT) by detecting the presence of type III Carbonic Anhydrase, CAIII, in a urine sample of a subject. The method is used for diagnosis and to monitor the progress of a condition, and to determine the efficacy of a treatment.

### FIELD OF THE INVENTION

The present invention relates to the field of diagnosis. In particular, the invention provides a method for diagnosing conditions related to a dysfunction of the renal proximal tubule (RPT) by using a new urinary biomarker called type III Carbonic Anhydrase. The invention further provides a diagnostic kit for diagnosing conditions related to a dysfunction of the RPT. In addition, the invention provides methods for identifying agents useful in the treatment of said conditions, and methods for monitoring the efficacy of a treatment for said conditions.

### BACKGROUND

The epithelial cells lining the RPT segment of the kidney play an essential role in reabsorbing ions, glucose, and amino acids from the primitive urine filtrated by the glomeruli. In particular, RPT cells avidly reabsorb several grams of albumin and low-molecular-weight (LMW) proteins that are daily filtered, through receptor-mediated endocytosis. This endocytic pathway, one of the most active of the body, involves two multiligand-binding receptors, megalin and cubilin, that are abundantly expressed at the brush border of RPT cells. Ligand binding and interactions between both receptors induce their internalization into coated vesicles at the apical membrane of RPT cells and their subsequent delivery to endosomes and lysosomes for ligand processing and receptor recycling. This endocytic trafficking is dependent on a progressive acidification from early to late endosomes and finally to lysosomes, a process that is driven by the vacuolar H⁺-ATPase (V-ATPase) complex coupled to a Cl⁻ conductance in order to dissipate the electrical gradient.

RPT dysfunction can be either inherited or acquired. The generalized RPT dysfunction, also named “renal Fanconi syndrome”, is a severe condition associated with variable degrees of solute (phosphate, glucose, amino acids, bicarbonate, salt) wasting, polyuria, hypercalciuria, and LMW proteinuria, that can lead to growth retardation, osteomalacia, rickets, nephrocalcinosis, and renal failure.

Recent insights into the pathophysiology of rare inherited RPT dysfunctions pointed to the importance of receptor-mediated endocytosis in the process. Inactivating mutations in the CLCN5 gene, which encodes the endosomal Cl—/H+ exchanger CIC-5, are associated with Dent's disease, an X-linked renal Fanconi syndrome characterized by LMW proteinuria and hypercalciuria, associated with glucosuria, amino-aciduria, phosphaturia, nephrocalcinosis, and nephrolithiasis. CIC-5 is primarily localized in the endosomes of RPT cells, where it co-distributes and is functionally linked with the V-ATPase. Genetic inactivation of Clcn5 in mouse causes renal tubular defects that mimic human Dent's disease, including severe RPT dysfunction with impaired endocytosis and trafficking defects. Likewise, the functional loss of cubilin in lmerslund-Grasbeck disease, as well as the genetic inactivation of megalin in mouse, lead to defective RPT reabsorption with increased urinary excretion of LMW proteins.

The biochemical and metabolic outcomes of RPT cellular dysfunction, as well as the potential adaptative mechanisms remain poorly understood. Recently, Wilmer et al. have reported an increased oxidative stress and altered redox status in RPT cells cultured from the urine of patients with cystinosis, the most frequent cause of inborn RPT dysfunction. These observations suggest that RPT dysfunction may be associated with increased solicitation of cell oxidative defences.

The early diagnosis of RPT dysfunction is essential for early and efficient treatment. Nowadays the diagnosis of RPT dysfunction relies on blood and urine analyses. Urinanalyses mostly use solute markers which are freely filtered by the glomeruli and normally reabsorbed by RPT cells, such as glucose, β₂-microglobulin, uric acid, aminoacids, and phosphate. Thus far no specific marker of the RPT cell damage itself has been clearly described. Rarely, detection of RPT dysfunction requires a renal biopsy, so involving a surgical procedure.

In addition, there is an urgent need to allow selection of appropriate agent(s) for therapeutic and/or prophylactic treatments of RPT dysfunction. The prediction of drug responsiveness phenotype of one given patient to one given drug (efficiency, dosing, adverse effects, etc) remains poor, thereby hampering the therapy itself.

In view of the above, it is clear that there remains a need in the art for a method which enables early and sensitive detection of RPT diseases.

Jouret et al. 2006 (Nephron physiology, vol 104, p. 43-44) reported the presence of type III carbonic anhydrase (CAIII), a kidney CA isozyme, with a distribution restricted to scattered proximal tubule (PT) cells, and suggested that this isozyme might protect RPT cells from oxidative damage. Induction of CAIII, in association with other cellular markers of cell proliferation and oxidative stress, was observed in kidney biopsies from a Dent's disease patient and in Clcn5 KO mouse kidneys using RT-PCR and immunoblotting analyses.

Rondeau et al. 2005 (Néphrologie & Thérapeutique, 1, 2006-209) indicate that Dent's disease involves proximal tubule dysfunction. The analyses of kidney biopsies from a subject (human or mouse) having Dent's disease showed that the expression of distinct cellular markers of cell proliferation and oxidative stress, as well as the one of CAIII, were increased.

In Jouret et al. 2006 and Rondeau et al. 2005 the authors have investigated the metabolic outcomes of proximal tubule dysfunction using a mouse model of Dent's disease, as well as a kidney biopsy of a patient with Dent's disease. They have demonstrated that proximal tubule deficiency caused by the functional loss of CIC-5 was associated with accelerated cell turnover and oxidative stress. AFLP-derived procedure, which compares mRNA abundance between two samples, showed that Car3 mRNA expression was induced in 010-5-deficient kidneys. This was confirmed at the mRNA and protein levels by real-time RT-PCR and immunoblotting, respectively. All data were strictly obtained from kidney biopsies.

US 2002/0177241 discloses methods useful to assay a sample, e.g. a urine sample, to detect the presence or relative levels therein of first and second analytes. The disclosed assays can for instance be used to determine the level of a first analyte, e.g. a cardiac marker such as myoglobine, in a sample and a second analyte, e.g. carbonic anhydrase III which is released from damaged skeletal muscle along with myoglobin. It is however noted that the disclosed analyses involve the combined detection of CAIII and myoglobin and that the disclosed methods do not enable single detection of CAIII in the absence of myoglobin.

Furthermore, this US application suggests that myoglobin/CAIII pair abundance in urine might be used to characterize in vivo kidney damage, i.e. glomerular diseases. However, such condition is different from and not related to a dysfunction of RPT. Moreover, the myoglobin/CA3 analyte pair can not be regarded as useful for determining in vivo the effective filtering capacity of the kidney for the following reasons. Basically, kidney function depends on plasma filtration through the glomerular membrane and selective tubular adjustments (absorption or secretion) of the filtrate to form the definitive urine. The glomerular filtration of plasma proteins depends on their size and their electrical charge. The physiological threshold for unrestrictive glomerular filtration of plasma proteins (independently of their charge) has been estimated at 69 kDa, which is the molecular weight of albumin. In other words, plasma proteins with a molecular weight higher than 69 kDa do not cross the glomerular membrane, whereas low-molecular-weight proteins (<69 kDa) are freely filtered. Pathological conditions affecting the glomerulus induce changes of glomerular pore size, resulting in the urinary excretion of high-molecular-weight proteins (>69 kDa). The molecular weights of myoglobin and CAIII are known to be around 17 kDa and 27 kDa, respectively. Therefore, the glomerular filtration of both proteins is not influenced by physiological or pathological changes in glomerular pore size, and the myoglobin/CAIII analyte pair can not be regarded as useful for determining in vivo the effective filtering capacity of the kidney.

The present invention aims to provide a urinary biomarker which enables early and sensitive detection of RPT diseases, and which overcomes at least some of the above-mentioned problems of known makers.

In addition, the present invention aims to provide a method for diagnosis.

Another object of the present invention is to provide a method for choosing or monitoring the efficacy of various treatments for RPT disorders.

### SUMMARY OF THE INVENTION

One embodiment of the invention is a method for determining a condition related to dysfunction of the renal proximal tubule (RPT) in a subject, comprising detecting the presence of type III Carbonic Anhydrase, CAIII, in a urine sample of said subject. In particular, the invention is directed to a method for determining a pathology causing or a condition related to renal proximal tubule (RPT) dysfunction in a subject the comprising measuring the presence of type III Carbonic Anhydrase, CAIII, in a urine sample of said subject and determining said pathology or said condition when said measured presence is different from the measured presence in a urine sample of a healthy subject.

CA-III or CAIII as used herein both refer to type III carbonic anhydrase. The term “presence of CAIII” as used herein is intended to encompass concentration of CAIII as well as a CAIII enzyme activity. In a preferred embodiment, measuring the presence of CAIII is therefore performed by measuring the concentration of CAIII in a sample. In another preferred embodiment, measuring the presence of CAIII is performed by measuring CAIII enzyme activity in a sample.

Another embodiment of the invention is a method as described above comprising the steps of:

(i) obtaining a urine sample from a subject;
(ii) measuring the concentration of CAIII in the sample;
(iii) comparing the concentration of CAIII in the sample with the concentration of CAIII in a healthy subject; and
(vi) determining a condition related to dysfunction of the RPT when the concentration of CAIII in the sample is different, and preferably at least 10% different, from that measured in a sample from a healthy subject.

Another embodiment of the invention is a method as described above comprising the steps of:

(i) obtaining a urine sample from a subject;
(ii) measuring the concentration of CAIII in the sample;
(iii) determining a condition related to dysfunction of the RPT when detectable CAIII is present in the sample or concentration of CAIII in the sample is greater than or equal to a threshold concentration.

Another embodiment of the invention is a method as described above wherein said threshold value is between 1 pM and 1 mM.

In yet another embodiment of the invention is a method as described above comprising the steps of:

(i) obtaining a urine sample from a subject;
(ii) measuring the CAIII enzyme activity in the sample;
(iii) comparing the CAIII enzyme activity in the sample with the CAIII enzyme activity in a healthy subject; and
(vi) determining a condition related to dysfunction of the RPT when the CAIII enzyme activity in the sample is different, and preferably at least 10% different, from that measured in a sample from a healthy subject.

Preferably said method as described above comprises the steps of:

(i) obtaining a urine sample from a subject;
(ii) measuring the CAIII enzyme activity in the sample;
(iii) determining a condition related to dysfunction of the RPT when the CAIII enzyme activity in the sample is greater than or equal to a threshold enzyme activity.

Another embodiment of the invention is a method as described above comprising the steps of:

(i) obtaining a urine sample from a subject;
(ii) detecting the presence of CAIII the sample;
(vi) determining a condition related to dysfunction of the RPT when CAIII is detected in the sample.

Another embodiment of the invention is a method for monitoring the progress of a pathology causing or a condition related to renal proximal tubule (RPT) dysfunction in a subject by monitoring the presence, i.e. the concentration or the enzyme activity of CAIII, in two or more urine samples taken at different intervals.

Another embodiment of the invention is a method as described above, comprising the steps of:

(i) obtaining two or more urine samples from a subject, taken at different time intervals;
(ii) measuring the concentration of CAIII in each sample;
(iii) determining the progress of a condition related to dysfunction of the RPT by comparing the concentrations of CAIII in the measured samples over time.

Another embodiment of the invention is a method as described above, comprising the steps of:

(i) obtaining two or more urine samples from a subject, taken at different time intervals;
(ii) measuring the CAIII enzyme activity in each sample;
(iii) determining the progress of a condition related to dysfunction of the RPT by comparing the CAIII enzyme activities in the measured samples over time.

Another embodiment of the invention is a method for monitoring the efficacy of a treatment of a pathology causing or a condition related to renal proximal tubule (RPT) dysfunction in a subject by measuring the presence of, and for instance detecting the level of, CAIII in a urine sample taken before, after and optionally during treatment. In a preferred embodiment measuring said presence is performed by measuring the concentration of CAIII in said samples. In another preferred embodiment measuring said presence is performed by measuring CAIII enzyme activity in said samples.

Another embodiment of the invention is a method as described above, comprising the steps of:

(i) obtaining a pre-administration urine sample from a subject prior to administration of the treatment;
(ii) measuring the presence such as the concentration or enzyme activity of CAIII in the pre-administration sample;
(iii) obtaining one or more post-administration urine samples from the subject;
(iv) measuring the presence such as the concentration or enzyme activity of CAIII the post-administration samples;
(v) comparing the presence such as the concentration or enzyme activity of CAIII in the pre-administration sample with presence such as the concentration or enzyme activity of CAIII in the post-administration sample or samples; and
(vi) determining the efficacy of a treatment.

Another embodiment of the invention is a method as described above, further comprising the step of altering the treatment to improve the effect of the treatment thereof, in particular to decrease the concentration or enzyme activity of CAIII in said post-administration samples.

Another embodiment of the invention is a method as described above, wherein the concentration or presence of CAIII in a sample is measured by using a CAIII specific probe. Another embodiment of the invention is a method as described above, wherein said CAIII probe is an antibody directed against CAIII or a fragment thereof. Another embodiment of the invention is a method as described above, wherein said antibody is a polyclonal antibody, monoclonal antibody, humanised or chimeric antibody, engineered antibody, or biologically functional antibody fragments sufficient for binding to CAIII. Another embodiment of the invention is a method as described above, wherein said antibody is mouse monoclonal antibody clone 2CA-4. Another embodiment of the invention is a method as described above wherein said CAIII human CAIII, a polypeptide having the sequence represented by SEQ ID NO: 1, or a fragment thereof.

Another embodiment of the invention is a method as described above, wherein the concentration or presence of CAIII is measured using any of biochemical assay, immunoassay, surface plasmon resonance, fluorescence resonance energy transfer, bioluminescence resonance energy transfer or quenching.

Another embodiment of the invention is a method as described above, wherein enzyme activity of CAIII is measured.

Another embodiment of the invention is a method as described above, wherein said condition is Fanconi syndrome or Dent's disease.

Another embodiment of the invention is a method as described above, wherein said condition is nephrotoxicity.

Another embodiment of the invention is a method as described above, wherein said arises from ingestion or infusion of heavy metals, chemotherapy agents, toxic drugs, poisons, pollutants, toxins or after injection with an iodinated contrast dye.

Another embodiment of the invention is a method as described above, wherein said condition is as a result of a physical renal injury.

Another embodiment of the invention is a method as described above, wherein said condition is renal or kidney failure or dysfunction.

Another embodiment of the invention is a method as described above, wherein said condition is acute renal failure include sepsis, shock, trauma, kidney stones, kidney infection, drug toxicity, poisons or toxins, or after injection with an iodinated contrast dye.

Another embodiment of the invention is a method as described above, wherein said condition is chronic renal failure, long-standing hypertension, diabetes, congestive heart failure, lupus, or sickle cell anemia.

Another embodiment of the invention is a method as described above, wherein said condition is inherited or acquired.

Another embodiment of the invention is a method as described above, further comprising detecting the presence of proteins and sugar in the urine.

Another embodiment of the invention is a use of CAIII as a urinary biomarker.

Another embodiment of the invention is a use of CAIII for diagnosing a condition related to dysfunction of the RPT in a subject.

Another embodiment of the invention is a device for determining a condition related to dysfunction of the RPT in a subject comprising means for determining the concentration and/or presence of CAIII in said urine sample.

Another embodiment of the invention is a device as described above, wherein said means comprise at least one CAIII specific probe.

Another embodiment of the invention is a device as described above, wherein said CAIII specific probe is as defined above.

Another embodiment of the invention is a device as described above, comprising a solid support whereby said CAIII is immobilised thereon.

Another embodiment of the invention is a device as described above, wherein said solid support comprises:
fluid capillary properties:
a distal (3) and proximal end (2),
a sample application zone (4) in the vicinity of the proximal end (2),
a reaction zone (5) distal to the sample application zone (4),
a detection zone (6) distal to the reaction zone (5),
where the reaction zone (5) is disposed with CAIII probe labelled with detection agent, that can migrate towards the distal end (3) in a flow of fluid by capillary action,
where the detection zone (6) comprises said immobilised CAIII probe that can capture CAIII.

Another embodiment of the invention is a device as described above, housed in a cartridge (20) watertight against urine, having an opening (21) to provide access to the application zone (4) in proximal end (2), and another opening (22) to enable reading of detection zone (6) close to the distal end (3).

Another embodiment of the invention is a device as described above, wherein said cartridge (20) is disposed with a sensor code (23) for communicating with a reading device.

In another embodiment, the invention relates to a device for determining a pathology causing or a condition related to renal proximal tubule (RPT) dysfunction in a subject comprising a reagent strip wherein said strip comprises a solid support provided with at least one test pad for measuring the presence of CAIII in an urine sample. Preferably said test pad comprises a carrier matrix incorporating a reagent composition capable of interacting with CAIII to produce a measurable response. In yet another embodiment a device is disclosed wherein said solid support further comprises one or more test pads for measuring the presence of one or more analytes selected from the group comprising proteins, blood, leukocytes, nitrite, glucose, ketones, creatinine, albumin, bilirubin, urobilinogen, and/or pH test pad and/or a test pad for measuring specific gravity.

In still another embodiment, the invention discloses a test pad for measuring the presence of CAIII in a urine sample, and preferably a test pad for measuring the concentration or the enzyme activity of CAIII in a urine sample. Preferably a test pad is disclosed wherein said pad comprises a carrier matrix incorporating a reagent composition capable of interacting with CAIII to produce a measurable response, e.g. concentration or enzyme activity. In another preferred embodiment the invention discloses a test pad for use in a reagent strip.

Another embodiment of the invention is a kit comprising a device as defined above and a urine sample container and/or control standards comprising CAIII. In particular, the invention relates to a kit for determining a condition related to dysfunction of the RPT comprising:
a solid support provided with means for determining the concentration and/or enzyme activity of CAIII in a urine sample, whereby said means comprise at least one CAIII specific probe,
control standards comprising CAIII, and
an urine sample container.

The present invention relates to methods for the diagnostic and monitoring of RPT disorders, i.e. injuries or toxicities, and to kits for diagnosing renal toxicity. In particular, the invention relates to the use of a urinary biomarker to determine renal disorders even before the disorder is demonstrated by histopathology examination, and/or to help choosing or monitoring the efficacy of various treatments for renal disorders. The present invention further provides methods and kits for diagnosing a pathology causing or a condition related to renal proximal tubule (RPT) dysfunction in a subject, for preventive screening of subjects such as school children or working people, for a pathology causing or a condition related to renal proximal tubule (RPT) dysfunction, or for monitoring renal or kidney transplantation(s) in a subject.

### DETAILED DESCRIPTION OF THE FIGURES

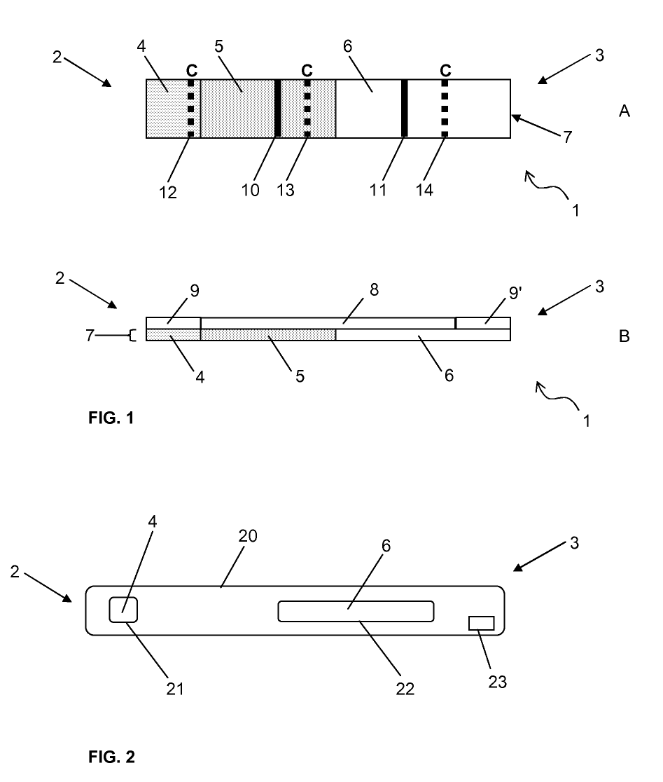

FIG. 1: Plan (A) and side view (B) of a test strip according to the invention.

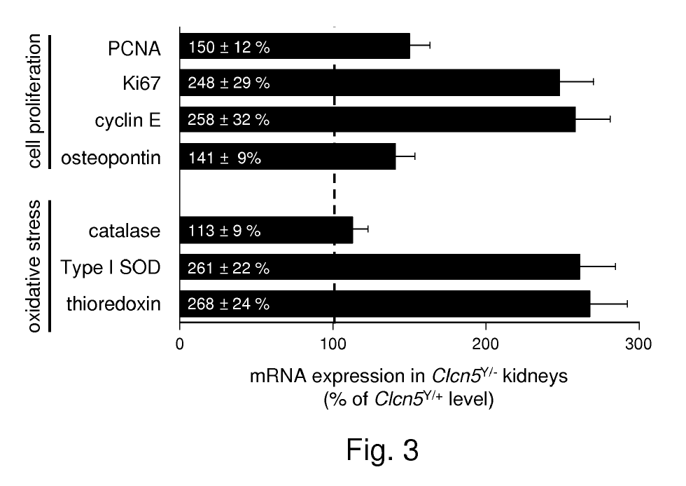

FIG. 2: Plan view of a test cartridge according to the invention

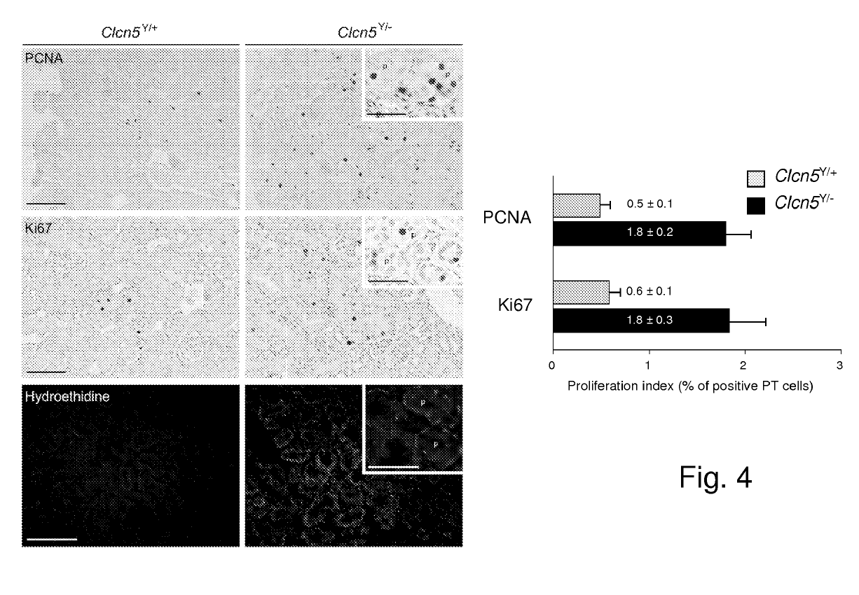

FIG. 3: Real-time RT-PCR analyses of cell proliferation and oxidative stress markers, such as PCNA, Ki67, cyclin E osteopontin, type I superoxide dismutase (SOD) and thioredoxin in kidneys from Clcn5^{Y/-} vs. Clcn5^{Y/2}-week-old mice (n=6 pairs). The mRNA levels were adjusted to GAPDH before quantification, and calculated upon the formula: Efficiency ^{ΔΔCt}. The Clcn5^{Y/-} kidneys show an increased expression of both cell proliferation and oxidative stress markers. Values are presented as mean ratios±SD, with Clcn5^{Y/+} level set at 100%; * p<0.05.

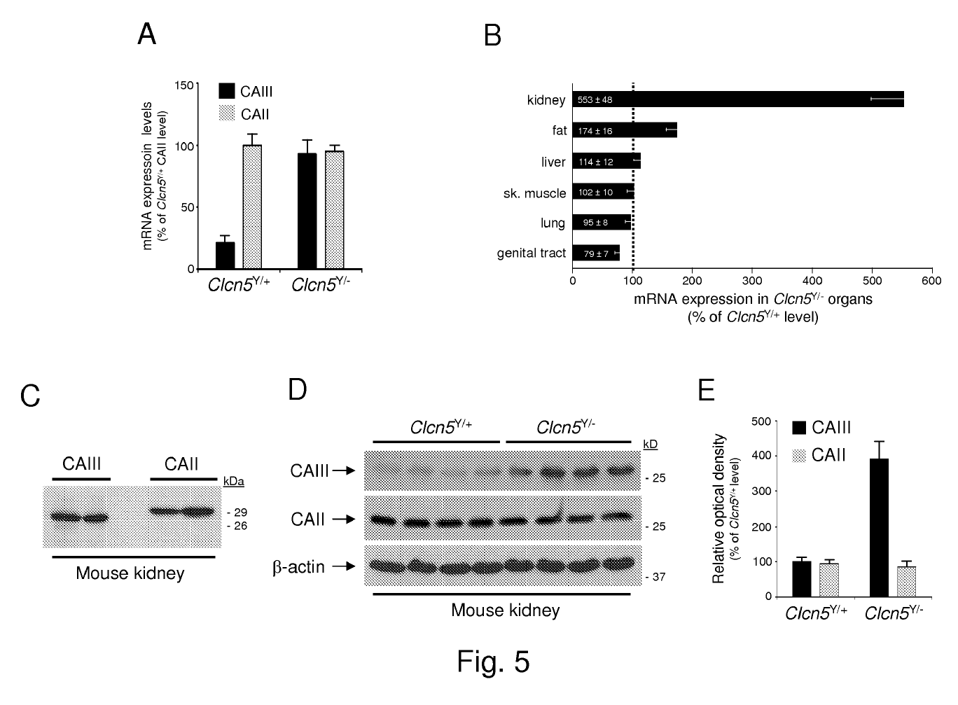

FIG. 4: Immunohistochemistry slides comparing PCNA-, Ki67- and ethidium bromide-positive cells in CIC-5 deficient and non-deficient kidneys (left) and proliferation indices for the same (right). Immunostaining for proliferation markers, PCNA and Ki67, and measurement of superoxide anion generation in Clcn5^{Y/+} and Clcn5^{Y/-} kidneys. Counting of PCNA- and Ki67-positive cells along PT (p) indicates a ˜3-fold increase of proliferating PT cells in Clcn5^{Y/-} vs. Clcn5^{Y/+} kidneys (n=4 pairs). Values are presented as means±SD; * p<0.05. The detection of red fluorescent ethidium bromide shows a positive signal in Clcn5^{Y/-} PT (p), in strong contrast to Clcn5^{Y/+} samples (n=3 pairs). Bars: 100 μm (insets, 50 μm).

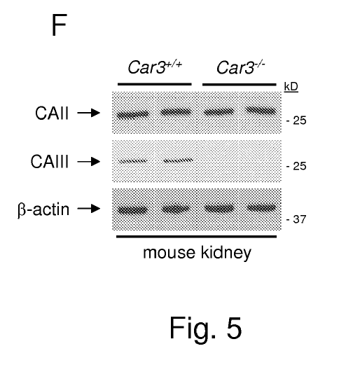

FIG. 5A: Results of quantitative real-time RT-PCR to compare the mRNA expression of CAIII and CAII in Clcn5Y/− and Clcn5Y/+ kidneys. Real-time RT-PCR quantification of mRNA expression of type III and II CA isozymes in Clcn5^{Y/-} vs. Clcn5^{Y/+} kidneys (n=6 pairs). The mRNA levels were adjusted to GAPDH and then compared between Clcn5^{Y/-} and Clcn5^{Y/+} samples, using the formula: Ratio=2^{-ΔΔCt}. In normal mouse kidneys, CAIII mRNA expression represents ˜20% of CAII. By contrast, in Clcn5^{Y/-} samples, CAIII expression is ˜5 times increased, with no changes in CAII level.

FIG. 5B: Levels of CAIII mRMA expression in Clcn5^{Y/-} organs. Real-time RT-PCR quantification of CAIII mRNA expression in Clcn5^{Y/-} vs. Clcn5^{Y/+} kidneys, epididymal fat, liver, skeletal muscle (vastus lateralis), lung and male genital tract (n=6 pairs). After adjustment of mRNA levels to the reporter gene GAPDH, CAIII mRNA quantification was compared between Clcn5^{Y/-} and Clcn5^{Y/+} samples, using the formula: Ratio=2^{-ΔCt}. The induction of CAIII caused by CIC-5 deficiency mostly involves kidneys, with a trend in epididymal fat and no significant changes in other organs.

FIG. 5C: Immunoblotting analysis showing the absence of antibody cross-reactivity between the two isozymes of CAIII. Twenty μg of cytosolic proteins from total kidneys (n=2 wild-type mice) were separated by SDS-PAGE and blotted onto nitrocellulose membrane. Anti-CAII antibodies (1/2000) detected a unique band around ˜29 kD, whereas CAIII was identified by anti-CAIII antibodies (1/1000) at a slightly lower molecular weight (˜27 kD), without cross-reactivity.

FIG. 5D: Immunoblotting analysis showing levels of CAIII and CAII in 12-week-old CIC-5 deficient and non-deficient kidneys. FIG. 5E: Optical density analyses of the results obtained in 5D. Panels D-E. Representative immunoblotting for CAII and CAIII in Clcn5^{Y/+} and Clcn5^{Y/-} kidneys. Twenty μg of cytosolic proteins were loaded in each lane. Blots were probed as in (C), and after stripping, for β-actin (1/10,000). Densitometry analyses show that CAIII expression is ˜4-fold higher in Clcn5^{Y/-} kidneys than in controls (385%±43 of Clcn5^{Y/+}, n=4 pairs of mice), whereas CAII abundance is unchanged. (* p<0.05)

FIG. 5F: Characterization of anti-CAIII antibodies. Twenty μg of cytosolic proteins from total Car3^{+/+} and Car3^{-/-} kidneys (n=2 pairs of mice) were separated by SDS-PAGE and blotted onto nitrocellulose membrane, before incubation with anti-CAII (1/1000) or anti-CAIII (1/1000) affinity-purified antibodies. The anti-CAIII antibodies do not detect any signal in the Car3^{-/-} kidneys, whereas type II CA is detected with anti-CAM antibodies in both Car3^{+/+} and Car3^{-/-} samples. Loading control was performed after membrane stripping and incubation with monoclonal antibodies anti-β-actin (1/10,000).

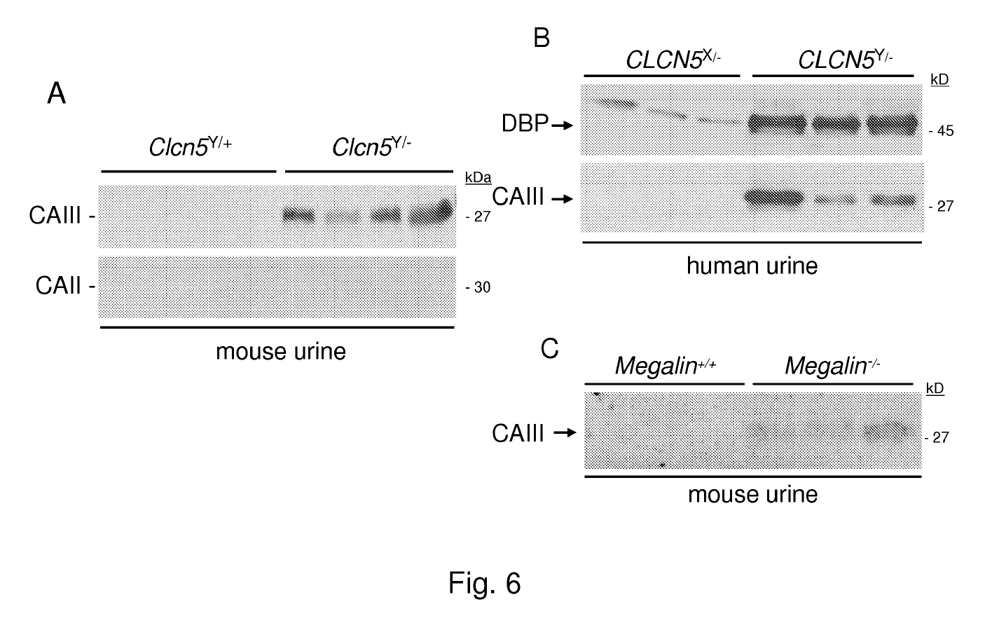

FIG. 6 Urinary excretion of CAIII. FIG. 6A: Immunoblotting analyses indicating a specific excretion of CAIII in the urine of the Clcn5^{Y/-} compared with Clcn5^{Y/+}. Urine samples from Clcn5^{Y/+} and Clcn5^{Y/-} mice (n=4 pairs of mice) were loaded on 14% PAGE, blotted onto nitrocellulose and incubated with anti-CAIII antibodies (1/1000). Loading volume was normalized for urine creatinine concentration. CAIII is exclusively detected in Clcn5^{Y/-} mouse urine. FIG. 6B: Urine samples from three patients with Dent's disease and their carrier mothers were loaded on 14% PAGE, blotted onto nitrocellulose and incubated with anti-DBP (1/1000) and anti-CAIII antibodies (1/1000). The low-molecular-weight protein, DBP, is barely detected in carriers and excreted in large amounts in patients. CAIII is only detected in patients with Dent's disease. Loading volume was normalized for urine creatinine concentration. FIG. 6C: urine samples from Lrp2^{+/+} and Lrp2^{-/-} (megalin) mice (n=3 pairs of mice) were loaded, according to creatinine concentration, on 14% PAGE, blotted onto nitrocellulose and incubated with anti-CAIII antibodies (1/1000). CAIII is specifically detected in megalin-deficient mouse urine.

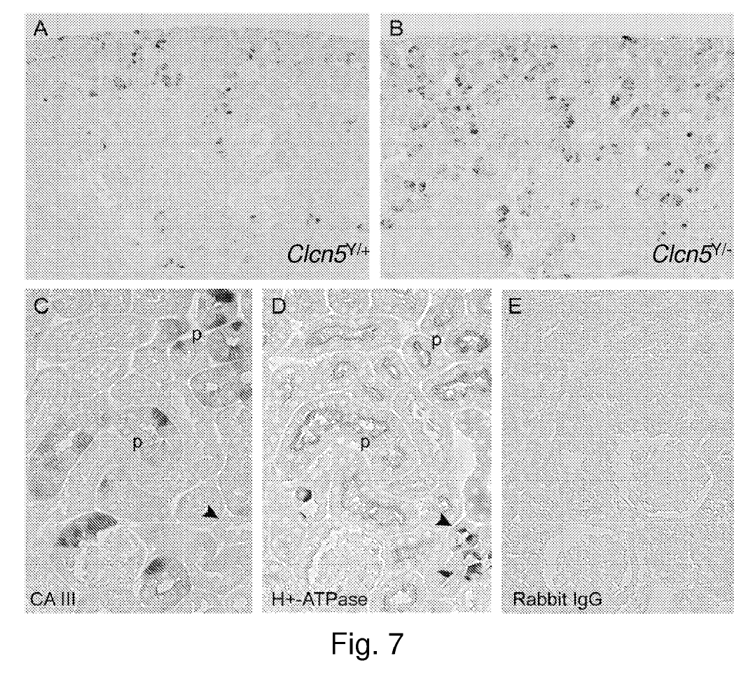

FIG. 7A to E: Immunohistochemistry slides showing the segmental distribution of CAIII in mouse kidney (low- and high-magnification). Immunostaining for CAIII (panels A-C), V-ATPase E1 subunit (panel D) in Clcn5^{Y/+} (panels A, C-E) and Clcn5^{Y/-} (panel B). C-D are serial sections (p, proximal tubule; g, glomerulus). In mouse control kidney, CAIII is present in some tubules in the outer cortex (A). In Clcn5^{Y/-} kidney, CAIII distribution includes both outer and inner cortices, with a ˜4-fold increased number of CAIII-positive cells (B). At higher magnification, CAIII is located in a subset of PT cells (C), identified by co-staining for the V-ATPase (D). The α-type intercalated cells of the collecting duct, which apically express the V-ATPase, are strictly negative for CAIII (C-D, arrowheads). No signal is detected after incubation with non-immune rabbit IgG (E). Bars: 100 μm (A-B); 50 μm (C-E).

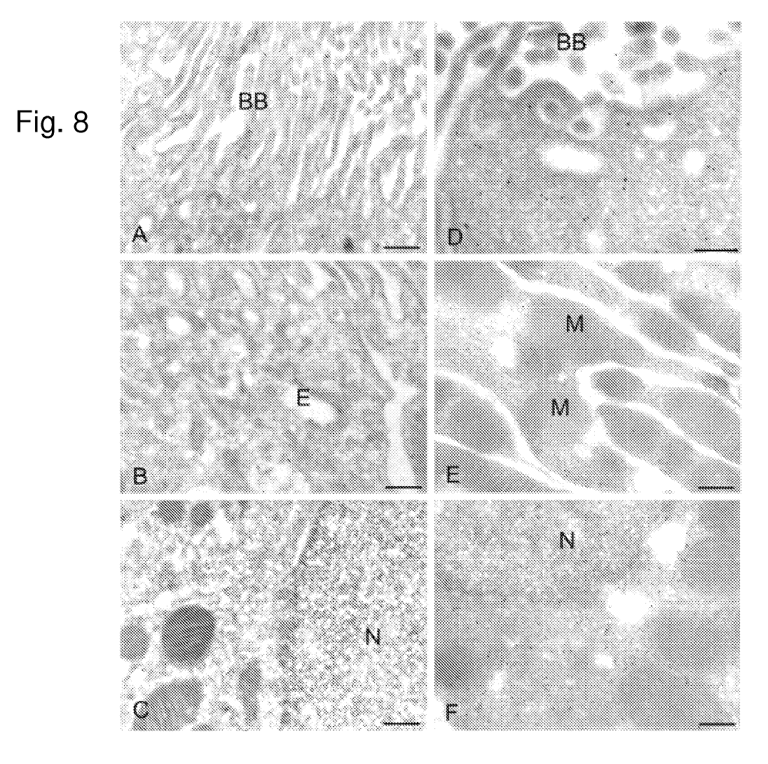

FIG. 8A to F: Immunogold analyses indicating the subcellular distribution of CAIII. EM immunocytochemistry for CAIII on ultrathin cryosections from renal cortex of Clcn5^{Y/-} (A-C) and Clcn5^{Y/-} mice (D-F). Labeling appears stronger in the Clcn5^{Y/-} samples than in controls. The labeling is mainly cytosolic, extending to the apical brush border (BB) microvilli (A, D). Nuclei (N) are also labelled (C, F) and a possible endosomal labeling (E in B) cannot be excluded. The very low signal in mitochondria (M in E) was considered to be background. Bars: A-C and F: 0.5 μm; D: 0.3 μm; and E: 0.8 μm.

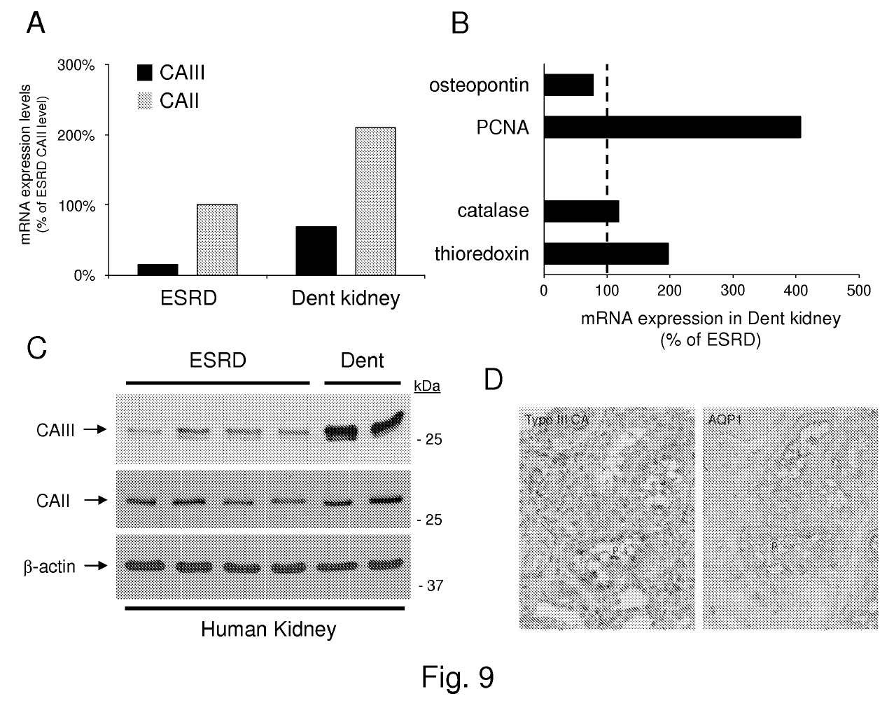

FIG. 9 A to B: mRNA quantification of CAIII and distinct markers of cell proliferation and oxidative stress in kidney samples from a patient with Dent's disease in comparison to 4 end-stage kidney samples taken as controls. Real-time RT-PCR quantification of mRNA expression of type III and II CA isozymes (A), osteopontin, PCNA, catalase and thioredoxin (B) in two cortical samples from one end-stage kidney from a patient with Dent's disease vs. four cortical samples obtained in end-stage kidneys of patients with an unrelated pathology (ESRD). The mRNA levels were adjusted to GAPDH, and quantified using the formula: Ratio=2^{-ΔΔXτ}. The CAIII mRNA expression is ˜4-fold higher in Dent's disease samples vs. ESRD controls, and associated with increased PCNA and thioredoxin mRNA levels. Note that CAII mRNA is also increased in the kidney samples of the patient with Dent's disease.

FIG. 9C: Comparative expression of CAIII protein in kidney samples from a patient with Dent's disease in comparison to 4 end-stage kidney samples taken as controls. Representative immunoblotting for CAIII and CAII isoforms in the human kidney samples described above (panel A). The blots were probed with antibodies against CAIII (1/1000) or CAII (1/2000), and after stripping, β-actin (1/10,000). A strong CAIII expression is observed in Dent's disease kidney.

FIG. 9D: Immunohistochemistry slides indicating the expression of CAIII in the human kidney. Immunostaining for CAIII (left) and aquaporin-1 (right) in human Dent's disease kidney. CAIII is located diffusely in some PT cells (p), identified by co-staining for the water channel AQP1.

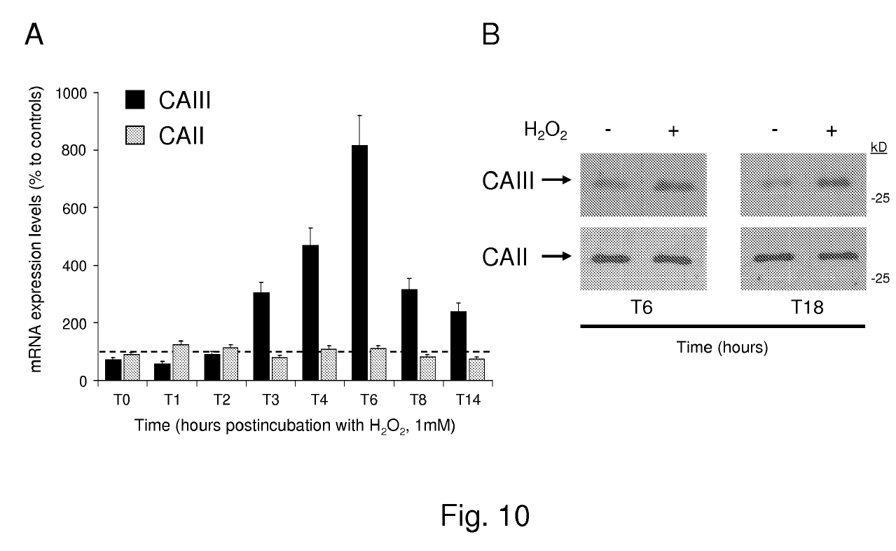

FIG. 10: Time-course of CAIII mRNA expression in HK-2 cells after H₂O₂ exposure; mRNA expression levels (A) of CAII and CAIII in HK-2 cells after incubation with H₂O₂. Real-time RT-PCR analyses of CAIII and CAII mRNA abundance in HK-2 cells after various periods of exposure to H₂O₂ (1 mM). Quantifications were done after adjustment to GAPDH mRNA levels and in comparison to time-matched controls. The expression of CAIII mRNA significantly increases from 3 h post H₂O₂ treatment, whereas no changes are observed in CAII. Values are presented as means±SD; * p<0.05. Immunoblotting analyses (B) for CAIII and CAII expression in HK-2 cells at various time-points following exposure to H₂O₂ (1 mM). Thirty μg proteins were loaded, blotted onto nitrocellulose membrane, and incubated with antibodies anti-CAIII (1/1000) or anti-CAIII (1/2000). In comparison to non-treated cells, H₂O₂-treated HK-2 cells show an increased expression of CAIII from 6 h posttreatment, with no changes in CAII expression.

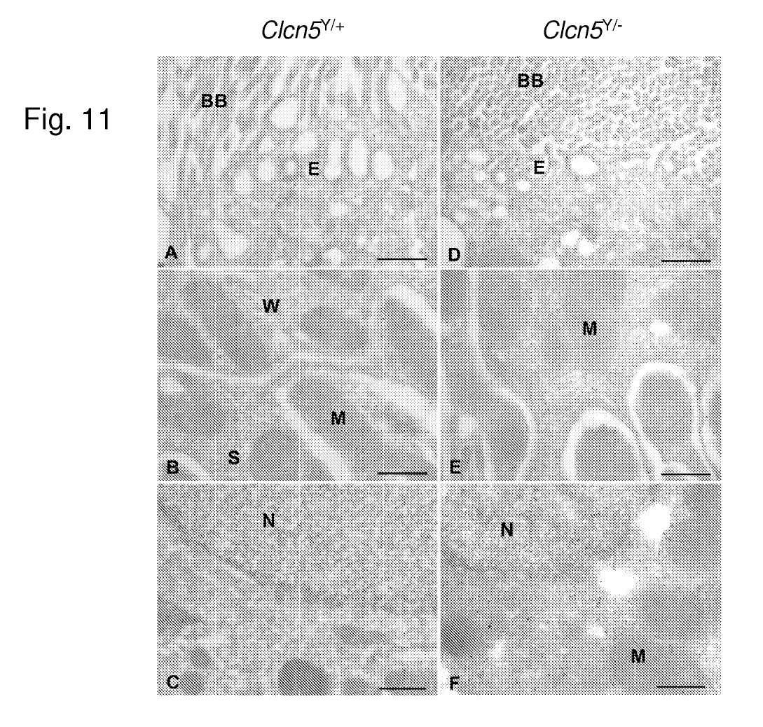

FIG. 11: illustrates subcellular distribution of CAIII in Clcn5Y/+ and Clcn5Y/− kidneys. EM immunocytochemistry for CAIII on ultrathin cryosections from renal cortex of Clcn5^{Y/+} (A-C) and Clcn5^{Y/-} mice (D-F). Labeling appears stronger in the Clcn5^{Y/-} samples than in controls. The labeling is mainly cytosolic, extending to the apical brush border (BB) microvilli (A, D). Nuclei (N) are also labelled (C, F) and a possible endosomal labeling (E in A and D) cannot be excluded. The very low signal in mitochondria (M) was considered to be background. W and S in B denote weakly and strongly labeled neighbor cells (compare to FIG. 7). Bars: 0.5 μm.

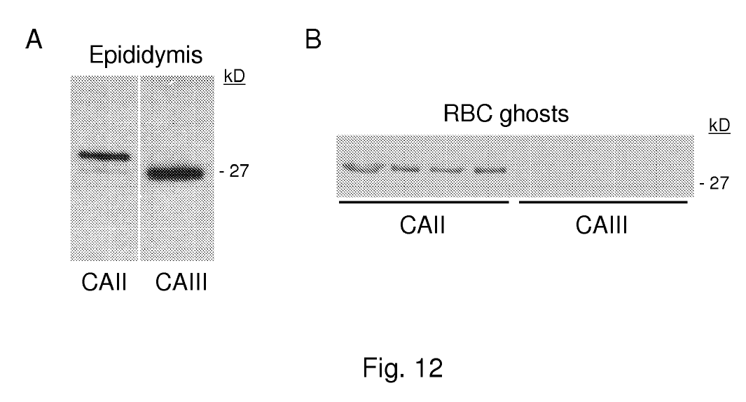

FIG. 12: illustrates the specificity of CAII and CAIII antibodies in extra-renal tissues, namely epididymis and red blood cells.

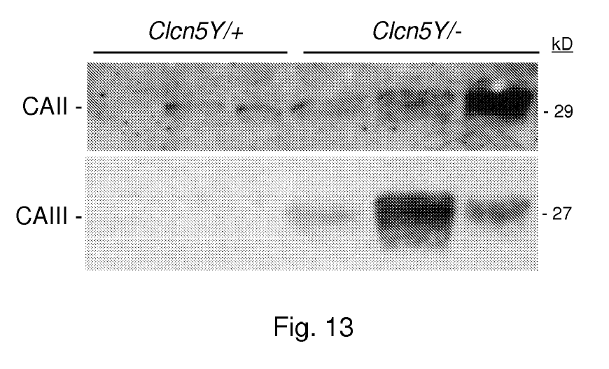

FIG. 13: shows detection of CAII and CAIII isozymes in urine samples of Clcn5^{Y/+} and Clcn5^{Y/-} mice.

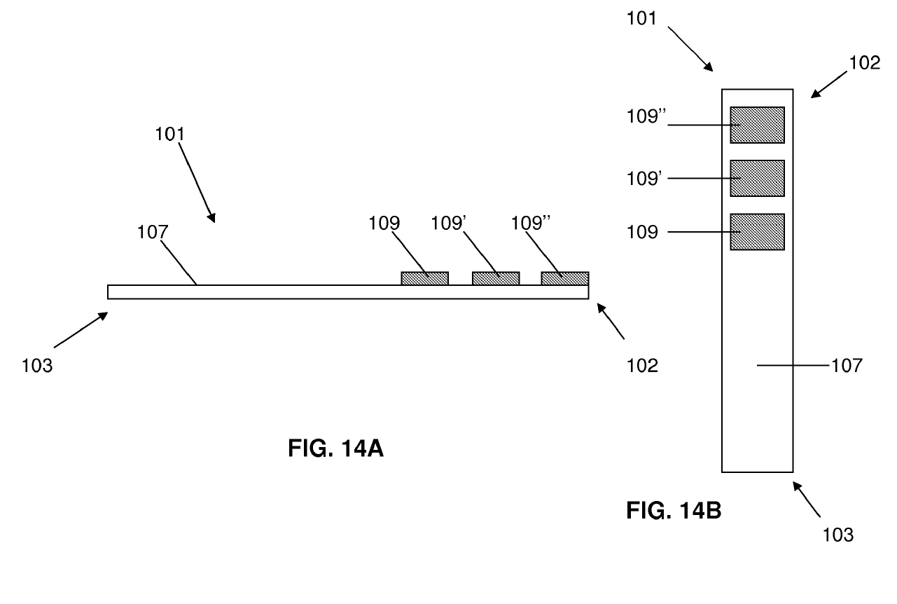

FIG. 14A-B shows a side view and a top view, respectively, of a reagent strip according to the invention comprising several test pads.

### DETAILED DESCRIPTION OF THE INVENTION

Unless defined otherwise, all technical and scientific terms used herein have the same meaning as is commonly understood by one of skill in the art. All publications referenced herein are incorporated by reference thereto. All United States patents and patent applications referenced herein are incorporated by reference herein in their entirety including the drawings.

The articles “a” and “an” are used herein to refer to one or to more than one, i.e. to at least one of the grammatical object of the article. By way of example, “a sample” means one sample or more than one sample.

Throughout this application, the term “about” is used to indicate that a value includes the standard deviation of error for the device or method being employed to determine the value.

The recitation of numerical ranges by endpoints includes all integer numbers and, where appropriate, fractions subsumed within that range (e.g. 1 to 5 can include 1, 2, 3, 4 when referring to, for example, a number of samples, and can also include 1.5, 2, 2.75 and 3.80, when referring to, for example, measurements). The recitation of end points also includes the end point values themselves (e.g. from 1.0 to 5.0 includes both 1.0 and 5.0).

The present invention relates to the finding by the inventors that a level of carbonic anhydrase III (CAIII) is locally modulated when there is a dysfunction of the renal proximal tubule (RPT) cells, and that the level of urinary excretion of CAIII is directly linked to RPT damage. In other words, a dysfunction of the RPT can be detected by measuring the level of CAIII in the urine. This technique relies on a specific RPT marker and avoids the need to take a biopsy of the kidney or RPT, which may be required to obtain a reliable diagnosis. Surprisingly the inventors have found that CAIII is excreted into the urine, by crossing the blood/urine barrier unlike many other disease markers which are filtered by the glomerular membrane before eventual RPT reabsorption. Furthermore, the level of CAIII production in dysfunctional RPT corresponds to the level detected in urine. This means elevated or reduced local CAIII levels are not distorted by any effect of storage in the bladder or modification by the urine. Furthermore, the present detection of CAIII is not distorted by any defects or deficiencies at the level of the renal filtering system. Additionally, they have found that CAIII in urine can be detected by specific binding assays i.e. there is little or no modification by the urine on CAIII at the molecular level. Furthermore the inventors have shown that single detection of CAIII can be used for diagnosis. CAIII need not be detected in conjunction with any other marker or analyte. The present invention is directed to a single measurement of CAIII in urine samples, without any other analyte. RPT dysfunction is associated with cell damage and direct shedding of CAIII into the urine, i.e. apparition of CAIII in the urine without the step of glomerular filtration. Therefore, CAIII detection in urine samples allows evaluating RPT dysfunction irrespective of any glomerular deficiency. This principle is clearly different from currently available tests for RPT dysfunction diagnosis which correspond to the measurement of the urinary concentration of plasma proteins originating from non-renal cells, β₂-microglobulin or Clara-cell protein CC16 for example. In physiological conditions, these low-molecular-weight proteins are freely filtered by the glomerular membrane and completely reabsorbed by RPT cells. In RPT dysfunction, such uptake of filtered plasma proteins does not occur, resulting in their urinary loss and detection. However, pathological processes affecting the glomerular composition (pore size) without RPT dysfunction, alter the ultrafiltration of plasma proteins, thereby interfering with diagnostic protein measurement. Such confusion result in “false-positive” cases. Thus, in contrast to currently available tests for RPT dysfunction, the quantification of CAIII in urine samples is directly dependent on RPT integrity, with no influence of the glomerular filtration and/or extra-renal organ function. Furthermore, the present invention is based on CAIII detection only in the urine, which per se is of pathological significance.

In contrast, assays as described in US 2002/0177241 are based on the ratio between serum abundance of a heart-specific marker, myoglobin, and a non-cardiac analyte, type III carbonic anhydrase (CAIII). In heart infarction, myoglobin is specifically released from damaged cardiac cells to the blood, with no participation of CAIII. In contrast, myoglobin and CAIII are co-released to the blood in case of skeletal muscle damage. A theoretical threshold allows distinguishing cardiac from non-cardiac muscle injury. Such assay needs the measure of the serum concentrations of both myoglobin and CAIII, and CAIII measurement is only used to isolate/calculate the fraction of serum myoglobin coming exclusively from the heart. Thus, CAIII in the urine is regarded as from muscle origin, and is primarily meant as a control for the specificity of myoglobin.

In Jouret et al. 2006 and Rondeau et al. 2005 the authors have investigated the metabolic outcomes of proximal tubule dysfunction using a mouse model of Dent's disease, as well as a kidney biopsy of a patient with Dent's disease. From these publications it can be concluded that CAIII participates to cell adaptation to the functional lack of CIC-5 and to the exposure to H₂O₂, like osteopontin, PCNA, Type I SOD and thioredoxin. However, there is no rationale to link CAIII induction in kidney biopsies from mice and patients with Dent's disease (paradigm of congenital RPT dysfunction) and its presence in corresponding urine samples. In this context it is also noted that other markers of cell turnover and oxidative stress, such as osteopontin PCNA, Ki67, Type I SOD or thioredoxin, are not detected in the urine. Thus, kidney expression of one given protein may not be unreservedly linked to its urine excretion, but urine presence of a marker depends on specific physio-pathological conditions/mechanisms related to its molecular nature. Restrictive analyses based on kidney biopsies do not support that CAIII induction in CIC-5-deficient renal tubule cells is associated with its presence in the urine. In addition, these data do not support or even suggest that CAIII measurement in urine samples might help diagnosing a disease or a condition related to inherited or acquired RPT dysfunction.

Summarised, it is not disclosed or even suggested in Jouret et al. 2006 and Rondeau et al. 2005 that (i) CAIII can be detected in urine samples; (ii) the urinary abundance of CAIII correlates with the severity of RPT dysfunction; and (iii) the urinary excretion of CAIII occurs in every cause, inherited or acquired, of RPT dysfunction. Moreover it cannot even be concluded from Jouret et al. 2006 and Rondeau et al. 2005 that the detection of CAIII in kidney biopsies represents a marker of RPT dysfunction.

The inventors have now found that the CAIII marker is extremely sensitive and allows the early diagnosis of RPT dysfunction such that a suitable therapy can be initiated at an early stage in the course of a disease. It also permits exquisite monitoring of a progress of a condition disease, and evaluation of its treatment.

In particular, the inventors show that CAIII is a useful urinary biomarker of RPT dysfunction, as they have measured its urinary excretion in distinct human and animal models of inherited and acquired RPT dysfunction. Their observations are at least partly based on immunoblotting analyses using well-characterized antibodies directed against CAIII and peroxidase-labelled secondary antibodies. This technique allowed to detect the presence of CAIII and to quantify its abundance in pathological urine samples.

Moreover, CAIII can be distinguished from other CA isozymes by specific biochemical properties and can be enzymatically detected and quantified in urine samples. The invention is therefore further directed to the use of two different methods of detection, i.e. antibody-based or enzymatic detection, to establish the concentration and the activity of CAIII in urine samples.

### Conditions Related to Dysfunction of the RPT

“Dysfunction of the renal proximal tubule (RPT)” as used herein is intended to refer to the abnormal functioning of the epithelial cells lining the RPT. A dysfunction of the RPT may be inherited or acquired. Abnormal functioning may include reduced functioning or malfunctioning or non-functioning. The present invention provides a method which permits to determine pathologies causing or conditions related to RPT dysfunction.

It shall be noted that the term “conditions” is used herein as a synonym for “pathologies” and is to be considered in its broadest sense, i.e. including environmental or physical situations as well as inherited diseases causing or resulting in RPT. In this context it shall be further noted that the present invention provides a method which permits to determine conditions related to or pathologies causing acquired as well as inherited RPT dysfunction. The term “condition related to a dysfunction of the RPT” refers to any pathology that gives rise to or causes, either directly or indirectly, an abnormal functioning of the epithelial cells lining the RPT, and thus a dysfunction of the RPT. A condition may be inherited or acquired.

In one embodiment the invention provides a method for determining an condition related to inherited renal proximal tubule (RPT) dysfunction, whereby said condition is selected from the group comprising COX deficiency, Cystinosis, Dent's disease (1), Dent's disease (2), Fanconi-Bickel syndrome, Fructosaemia, Galactosaemia, Imerslund-Gräsbeck disease, Lowe syndrome, Tyrosinaemia, von Gierke disease, Wilson disease, Type III MODY (maturity-onset diabetes of the young) diabetes, and cystic fibrosis. The table 1 presented below summarises conditions or pathologies causing inherited RPT dysfunction.

TABLE 1

| condition or | | | | |
|---|---|---|---|---|
| pathology | OMIM | Gene | Protein | Inheritance |
| COX deficiency | #220110 | MTC01-03 | Cytochrome c | AR |
| | | MTTS1 | oxidase | |
| | | COX10 | | |
| | | SC01-02 | | |
| Cystinosis | #219800 | CTNS (17p13) | Lysosomal cystine | AR |
| | | | transporter | |
| Dent's disease (1) | #300009 | CLCN5 (Xp11.22) | H⁺/Cl⁻exchanger | XR |
| Dent's disease (2) | #300555 | OCRL (Xq26.1) | PIP₂ 5-phosphatase | XR |
| Fanconi-Bickel | #227810 | GLUT2 (3q26.1-3) | Glucose transporter | AR |
| syndrome | | | GLUT2 | |
| Fructosaemia | +229600 | ALDOB (9q22.3) | Fructose- | AR |
| | | | bisphosphate | |
| | | | aldolase B | |
| Galactosaemia | #230400 | GALT (9p13) | Galactose 1- | AR |
| | | | phosphate | |
| | | | uridylyltransferase | |
| Imerslund- | #261100 | CUBN (10p12.1) | Cubilin | AR |
| Gräsbeck disease | | AMN (14q32) | Amnionless | |
| Lowe syndrome | #309000 | OCRL (Xq26.1) | PIP₂ 5-phosphatase | XR |
| Tyrosinaemia | +276700 | FAH (15q23-25) | Fumarylacetoacetase | AR |
| von Gierke disease | +232200 | G6PC (17q21) | Glucose 6- | AR |
| | | | phosphatase | |
| Wilson disease | #277900 | ATP7B (13q14.3-21.1) | Copper-transporting | AR |
| | | | ATPase 2 | |

A “number” symbol (#) indicates that the phenotype is not linked to a unique locus, whereas a “plus” sign (+) means that the entry associates a gene with a phenotype (AR: autosomal recessive; XR: X-linked recessive).

In another embodiment, the invention provides a method for determining a condition related to acquired renal proximal tubule (RPT) dysfunction. In a preferred embodiment a pathology causing or a condition related to acquired dysfunction of RPT is selected from the group comprising;
nephrotoxicity for instance due to the ingestion or infusion of toxic compounds and drugs such as heavy metals, aminoglycoside antibiotics, anti-retroviral drugs (e.g. azidothymidine), chemotherapy agents (e.g. ifosfamide, cisplatin), poisons, pollutants, toxins, etc.
renal injury,
acute or chronic renal or kidney failure,
multiple myeloma,
light chain deposition disease,
renal transplantation,
etc.

The list of acquired causes includes multiple myeloma, light chain deposition disease, and renal transplantation. In addition, various toxic compounds and drugs have been associated with PT defects, especially heavy metals such as cadmium, uranium, lead and mercury, aminoglycoside antibiotics, as well as some anti-retroviral drugs e.g. azidothymidine and chemotherapy with cytotoxic drugs, e.g. ifosfamide, cisplatin. Most of these compounds affect the endocytic/lysosomal system and the mitochondrial function, which might explain their particular toxicity for the PT.

A dysfunction of the RPT can thus also be a result of toxicity (nephrotoxicity) and can arise from ingestion or infusion of toxic compounds such as heavy metals, chemotherapy agents, toxic drugs, poisons, pollutants, toxins or after injection with an iodinated contrast dye (adverse effect) etc.

A dysfunction of the RPT can also be a result of a renal injury.

A dysfunction of the RPT can also be a result of a renal or kidney failure or dysfunction either sudden (acute) or slowly declining over time (chronic). Examples of situations/circumstances which give rise to acute renal failure include sepsis (infection), shock, trauma, kidney stones, kidney infection, drug toxicity, poisons or toxins, or after injection with an iodinated contrast dye (adverse effect). Examples of situations/circumstances which give rise to chronic renal failure include long-standing hypertension, diabetes, congestive heart failure, lupus, or sickle cell anemia. Both forms of renal failure result in a life-threatening metabolic derangement.

### Urine Sample

The urine sample as used herein is generally unprocessed urine. However, the invention includes urine to which common stabilizing additives such as anti-bacterial-growth agents or protein stabilizing agents have been added, as well as urine sediments and supernatant obtained by centrifuging urine. The volume of urine required to perform the assay will depend on the technique used to assay the amount of CAIII. The skilled person can readily perform tests to determine the sensitivity of the assay using control sample of CAIII, and adapt the volume of urine required accordingly. The subject may provide a urine sample in a regular urine sample bottle which typically holds between 25 to 100 ml urine.

Carbonic anhydrase III

The CAIII refers to full length human CAIII. According to an aspect of the invention, CAIII is a polypeptide having the sequence represent by SEQ ID NO: 1 in Table 1.

TABLE 1
Amino acid sequence of CAIII according to the
invention

SEQ ID NO: 1, amino acid sequence of human
CAIII (Accession no: NP_005172)
MAKEWGYASH NGPDHWHELF PNAKGENQSP VELHTKDIRH
DPSLQPWSVS YDGGSAKTIL NNGKTCRvVF DDTYDRSMLR
GGPLPGPYRL RQFHLHWGSS DDHGSEHTVD GVKYAAELHL
VHWNPKYNTF KEALKQRDGI AVIGIFLKIG HENGEFQIFL
DALDKIKTKG KEAPFTKFDP SCLFPACRDY WTYQGSFTTP
PCEECIVWLL LKEPMTVSSD QMAKLRSLLS SAENEPPVPL
VSNWRPPQPI NNRVVRASFK

CAIII also refers to a fragment of CAIII which has a unique property, allowing identification of the fragment as a fragment of CAIII. A unique property may be, for example, a unique sequence or unique reactivity with binding agent such as an antibody or probe.

According to one embodiment of the invention, a fragment of CAIII comprises one or more contiguous deletions from the C- or N-terminal end, or both. The number of deletions may be equal to or less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 amino acids. Preferably the number of deletions is between 1 and 30 amino acids.

### Detecting Presence of a Condition

One embodiment of the present invention is a method for diagnosing a condition related to dysfunction of the RPT in a subject by detecting the presence or level or enzyme activity of CAIII in a urine sample. The presence or level or enzyme activity may be compared with that of healthy subjects.

In a preferred embodiment, the present invention provides a method for determining a condition related to dysfunction of the RPT in a subject comprising the steps of:

(i) obtaining a urine sample from a subject;
(ii) measuring the concentration or enzyme activity of CAIII in the sample;
(iii) comparing the concentration or enzyme activity of CAIII in the sample to the concentration or enzyme activity of CAIII in a healthy subject sample; and
(vi) determining a condition related to dysfunction of the RPT when the concentration of CAIII in the sample is different from that measured in a sample from a healthy subject.

One embodiment of the present invention, a concentration of CAIII in a sample that is at least 10% (e.g. at least 20%, 30%, 40%, 50%, 60%, 70%, 80% or 90%) different from that measured in a sample from a healthy subject, identifies the subject as having a dysfunction of the RPT. The concentration of CAIII in the sample may be higher or lower than that in a healthy subject to indicate a dysfunction; preferably it is higher.

Another embodiment of the present invention, enzyme activity of CAIII in a sample that is at least 10% (e.g. at least 20%, 30%, 40%, 50%, 60%, 70%, 80% or 90%) different from that measured in a sample from a healthy subject, identifies the subject as having a dysfunction of the RPT. The enzyme activity of CAIII in the sample may be higher or lower than that in a healthy subject to indicate a dysfunction; preferably it is higher.

According to another preferred embodiment, the present invention provides a method for determining a condition related to dysfunction of the RPT in a subject comprising the steps of:

(i) obtaining a urine sample from a subject;
(ii) measuring the concentration of CAIII in the sample;
(iii) determining a condition related to dysfunction of the RPT when detectable CAIII is present in the sample or concentration of CAIII in the sample is greater than or equal to a threshold concentration.

The threshold concentration can be extremely low as the inventors found that no detectable CAIII is present in healthy subjects. According to one aspect of the invention, the threshold concentration is 1 pM, 5 pM, 10 pM, 50 pM, 100 pM, 500 μM, 1 nM, 5 nM, 10 nM, 50 nM, 100 nM, 500 nM, 1 μM, 5 μM, 10 μM, 50 μM, 100 μM, 500 μM, 1 mM, 5 mM, 10 mM, 50 mM, 100 mM, 500 mM; the concentration of CAIII in the sample a value in the range between any two of the aforementioned values; preferably it is between 1 μM and 1 mM.

According to another preferred embodiment, the present invention provides a method for determining a condition related to dysfunction of the RPT in a subject comprising the steps of:

(i) obtaining a urine sample from a subject;
(ii) measuring the CAIII enzyme activity in the sample;
(iii) determining a condition related to dysfunction of the RPT when the CAIII enzyme activity in the sample is greater than or equal to a threshold enzyme activity.

The inventors have found that a healthy subject may have no detectable CAIII in their urine. Therefore, a qualitative (yes/no) rather than a quantitative (concentration) measure of CAIII may only be necessary to determine the presence of a condition.

In a preferred embodiment, the present invention provides a method for determining a condition related to dysfunction of the RPT in a subject comprising the steps of:

(i) obtaining a urine sample from a subject;
(ii) detecting the presence of CAIII in the sample;
(vi) determining a condition related to dysfunction of the RPT when CAIII is detected in the sample.

The presence of CAIII in a sample, and/or concentration and/or enzymatic activity thereof can be determined using the binding assays described below.

### Monitoring Progress of a Disease

Another embodiment of the present invention is a method for monitoring the progress of a condition related to dysfunction of the RPT in a subject by monitoring the presence or concentration (level) or enzyme activity of CAIII in two or more urine samples taken over time intervals. The presence or level or enzyme activity may be compared with that of healthy subject.

The monitoring generally entails measuring the presence or level or enzyme activity of CAIII in urine sample from a subject, which sample is taken at regular periods e.g. over the course of a number of days, weeks or months. The presence or level or enzyme activity of CAIII in the urine sample over time can give an indication of whether a condition is improving, worsening or has stabilized.

In a preferred embodiment, the present invention provides a method for monitoring the progress of a condition related to dysfunction of the RPT in a subject comprising the steps of:

(i) obtaining two or more urine samples from a subject, taken at different time intervals;
(ii) measuring the concentration of CAIII in each sample;
(iii) determining the progress of a condition related to dysfunction of the RPT by comparing the concentrations of CAIII in the measured samples over time.

In another preferred embodiment, the present invention provides a method for monitoring the progress of a condition related to dysfunction of the RPT in a subject comprising the steps of:

(i) obtaining two or more urine samples from a subject, taken at different time intervals;
(ii) measuring the CAIII enzyme activity in each sample;
(iii) determining the progress of a condition related to dysfunction of the RPT by comparing the CAIII enzyme activities in the measured samples over time.

The time interval may be any which can give can give rise to a detectable change in the case of disease progression or retardation. The time interval can depend on the sensitivity of the measurement step. For example, a highly sensitive technique, with little background signals, might reveal small changes in the concentrations or CAIII enzyme activities of CAIII in the sample; consequently the time interval between sample can be short e.g. between 1 to 7 days. A less sensitive technique, on the other hand, would not reveal small changes, so necessitating larger time interval between samples e.g. between 1 to 3 weeks. According to one embodiment of the invention, the time interval between samples can be less than or equal to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 days or a value in the range between any two of the aforementioned values. Preferably the time interval is between 1 to 10 days.

The presence of CAIII in a sample and/or concentration and/or enzymatic activity thereof can be determined using assays as described below.

### Monitoring the Efficacy of a Treatment

Another embodiment of the present invention is a method for monitoring the efficacy of a treatment of a condition related to dysfunction of the RPT in a subject by measuring the presence of CAIII in a urine sample taken before, after and optionally during treatment. A reduction in the level or enzyme activity of CAIII will indicate an effective treatment.

Where the treatment comprises the administration of an agent (e.g., drug compounds, an agonist, antagonist, peptidomimetic, protein, peptide, nucleic acid, small molecule, or other drug candidate), the invention can be advantageously applied in clinical trials. For example, the effectiveness of an agent to affect the levels or enzyme activity of CAIII can be monitored in clinical trials of subjects receiving treatment for RPT dysfunction. Furthermore, the treatment can be altered according to the efficacy of the treatment.

In a preferred embodiment, the present invention provides a method for monitoring the effectiveness of treatment of a subject with an agent comprising the steps of:

(i) obtaining a pre-administration urine sample from a subject prior to administration of the agent;
(ii) measuring the concentration or enzyme activity of CAIII in the pre-administration sample;
(iii) obtaining one or more post-administration urine samples from the subject;
(iv) measuring the concentration or enzyme activity of CAIII in the post-administration samples;
(v) comparing the concentration or enzyme activity of CAIII in the pre-administration sample with the level of CAIII in the post-administration sample or samples; and
(vi) determining the efficacy of a treatment.

After determining the efficacy of a treatment, the treatment can be changed, for example, to improve the effect. This might entail altering an agent and/or administration thereof to the subject accordingly. For example, modified administration of the agent can be desirable to decrease the level or enzyme activity of CAIII to lower levels than detected, i.e., to increase the effectiveness of the agent. Alternatively, increased/decreased administration of the agent can be desirable to increase/decrease the effectiveness of the agent, respectively.

Another particular aspect of the present invention, a method is provided for both prophylactic and therapeutic methods of treating a subject having, or at risk of having, a kidney disorder or renal toxicity. Administration of a prophylactic agent can occur prior to the manifestation of symptoms characteristic of the kidney disorder, such that development of the kidney disorder is prevented or delayed in its progression. Examples of suitable therapeutic agents include, but are not limited to, antisense nucleotides, ribozymes, double-stranded RNAs, ligands, small molecules and antagonists.

The presence of CAIII in a sample and/or concentration and/or enzymatic activity thereof can be determined using assays as described below.

### Binding Assays for Detecting Level (Concentration) of CAIII

One embodiment of the present invention, the presence and/or concentration of CAIII in a sample is detected by using a CAIII probe specific for CAIII. Typically, CAIII probe and CAIII form a complex which causes a physical change (e.g. colour change, change in polarization) compared with the uncomplexed forms, which physical change can be detected. The magnitude of the change is usually in proportion to the quantity of complex. When the reaction is calibrated with standard concentrations of CAIII, the quantity of CAIII can be calculated by comparing the change with a standard. It is not always necessary to employ standard controls, for example, if, for example, the concentration of CAIII probe is known, and tight binding is assumed, it can be possible to calculate the concentration of CAIII. When a only qualitative result is needed, standard concentration controls may not be needed

As used herein, the binding between CAIII and CAIII probe refers to their physical association. The binding is generally specific, meaning it occurs with a Kd of 1 mM or less, generally in the range of 100 nM to 10 pM. For example, binding is specific if the Kd is 100 nM, 50 nM, 10 nM, 1 nM, 950 pM, 900 pM, 850 pM, 800 pM, 750 pM, 700 pM, 650 pM, 600 pM, 550 pM, 500 pM, 450 pM, 350 pM, 300 pM, 250 pM, 200 pM, 150 pM, 100 pM, 75 pM, 50 pM, 25 pM, 10 pM or less. Being specific can also mean it is unique, i.e. there is no cross-reactivity between the CAIII probe any other substance besides CAIII and a fragment thereof.

The measuring is typically performed under conditions permitting the binding between CAIII and a CAIII probe; this refers to conditions of, for example, temperature, salt concentration, pH and protein concentration under which binding will arise. Exact binding conditions will vary depending upon the nature of the assay, for example, whether the assay uses pure probe or only partially purified probe. Temperatures for binding can vary from 15 deg C. to 37 deg C., but will preferably be between room temperature and about 30 deg C.

One embodiment of the present invention, a concentration of CAIII in a sample that is at least 10% (e.g., at least 20%, 30%, 40%, 50%, 60%, 70%, 80% or 90%) different from that measured in a sample from a healthy subject, identifies the subject as having a dysfunction of the RPT. The concentration of CAIII in the sample may be higher or lower than that in a healthy subject to indicate a dysfunction; preferably it will be higher.

The measuring may be performed using any method known in the art. Preferably, the method selected from biochemical assay (e.g., solid phase assay), surface plasmon resonance, fluorescence resonance energy transfer (FRET), bioluminescence resonance energy transfer (BRET), fluorescence quenching, and fluorescence polarisation. Such techniques are well known and are described as follows.

### Biochemical Assays

Biochemical assays for determining the binding between two components generally rely on the immobilisation of one binding component, for example, on a membrane or other solid support, and exposure to the second binding component. After washing away excess of the second binding component, bound complex is detected by, for example, immunoassay, or by using labelled components (e.g., radio-labels, fluorescently labels, particulate labels). For example, a CAIII probe may be immobilised i.e. cannot be routinely washed off) not wash onto a nitrocellulose membrane and exposed to the sample. Bound CAIII can be detected using a particle-labeled antibody, or primary and optionally secondary antibody immunoassays to arrive at a concentration of AC-III present in the sample. The roles of a CAIII and CAIII probe may be switched; the skilled person may adapt the method so CAIII probe is applied to immobilised CAIII to determine binding.

By solid support herein is meant any solid support which is capable of immobilising components and/or samples. Such solid supports are known in the art and include, but are not limited to, nitrocellulose, glass slides, nylon, silane coated slides, nitrocellulose coated slides, plastics, ELISA trays, magnetic beads. A solid support may be capable of holding single spotted sample, multi-samples and/or micro-arrays etc.

Where an immunoassay is employed, such immunoassay methods include, but are not limited to, dot blotting, western blotting, competitive and noncompetitive protein binding assays, enzyme-linked immunosorbant assays (ELISA), immunohistochemistry, fluorescence activated cell sorting (FACS), and others commonly used and widely described in scientific, patent literature, or employed commercially.

Biochemical assays such as the ELISA may rely on a colour change to indicate the presence or absence of CAIII in a sample, and to provide an indication of concentration by virtue of intensity. Since the colour change can be read by eye, such assays can be performed without resorting to instrumentation to read the result. Biochemical assays, therefore, may be employed outside the laboratory, for example, in the doctors surgery, by a visiting healthcare worker or as a home testing kit.

### Enzymatic Assays

Amounts of enzymes can be measured n terms of activity, in enzyme units. Enzyme activity=moles of substrate converted per unit time=rate×reaction volume. Enzyme activity is a measure of the quantity of active enzyme present and is thus dependent on conditions. Enzyme activity is expressed in the SI unit katal, 1 katal=1 mol s⁻¹, or as 1 enzyme unit (EU)=1 μmol min⁻¹ (μ=micro, ×10⁻⁶). 1 U corresponds to 16.67 nanokatals. All enzyme assays measure either the consumption of substrate or production of product over time. Enzyme assays can be split into two groups according to their sampling method: continuous assays, where the assay gives a continuous reading of activity, and discontinuous assays, where samples are taken, the reaction stopped and then the concentration of substrates/products determined. Continuous assays may include spectrophotometric assays, colorimetric assays, fluorimetric assays and chemiluminescent assays. Discontinuous assays are when samples are taken from an enzyme reaction at intervals and the amount of product production or substrate consumption is measured in these samples. Discontinuous assays may include radiometric assays, and chromatographic assays

Another embodiment of the invention is a method as described above, wherein enzyme activity of CAIII is measured, by measuring for instance its CO₂ hydratase activity, its resistance to sulphonamide inhibitors, as well as its percarbonic anhydrase activity, according to techniques well known in the art, for instance disclosed
in Maren (J Phar Exp Ther 130: 26-29, 1960), (Anal. Biochem. 77, 552-561, 1977), wherein carbonic anhydrase activity was measured using the changing pH technique with a barbital buffer, a continuous flow of CO₂ and the addition of increasing amounts of diluted red blood cells. Units were expressed per milliliter of red cells (U/ml).
a publication wherein Carbonic anhydrase activity was determined in the diluted haemolysates and supernatant fluids at 25° C. by the method of Livesey (Anal. Biochem. 77, 552-561, 1977) in a stopped-flow rapid-kinetics cell (Hi-Tech Scientific, Salisbury, Wilts., U.K.), the operational unit of enzyme activity (MU) being as defined by Maren (Physiol. Rev. 47, 595-781, 1967).
a publication wherein CA activity in supernatant was analyzed using the electrometric delta pH assay (Henry RP Techniques for measuring carbonic anhydrase activity in vitro: the electrometric delta pH and pH stat methods. Pp. 119-125 in The Carbonic Anhydrases, S. J. Dodgson, R.E. Tashian, G. Gros, and N. D. Carter, eds. Plenum Publishing, New York. 1991).
or by continuous Spectrophotometric Rate Determination (see example 4)

In urine samples CAIII enzyme activity can be distinguished from other CA isozymes by specific biochemical properties. In particular, CAIII has a very low CO₂ hydratase activity (1% of CAII activity) and is resistant to sulphonamide (i.e. acetazolamide) inhibitors. Moreover, CAIII might act as a percarbonic anhydrase. Assays for determining CAIII activity can be based on determination of one or more of the above indicated activities or features.

In addition assays for determining CAIII activity can be based on the determination of CAII enzyme activity. In an example CAII activity can be determined by applying standard methods for determining CAII activity which have been described in the art, e.g. in biochemistry textbooks. CAIII activity is regarded as 1% of CAII activity.

### Devices for Measuring the Presence of CAIII in an Urine Sample

In another embodiment, the invention provides devices for determining a condition or pathology related to dysfunction of the RPT in a subject comprising means for measuring the presence of CAIII, and in particular for determining the concentration and/or enzymatic activity of CAIII, in a urine sample.

In one embodiment, the invention provides a dipstick. Such dipstick comprises a test strip allowing migration of an urine sample by capillary flow from one end of the strip where the sample is applied to the other end of such strip where presence of an analytes in said sample is measured.

In another embodiment, the invention provides a device comprising a reagent strip. Such reagent strip comprises one or more test pads which when wetted with the urine sample, provide a color change in the presence of an analyte.

### A. Test Strip and Cartridge—Dipstick

Another way to perform a biochemical assay is to use a test-strip and labelled antibodies which combination does require any washing of the membrane. The test strip is well known, for example, in the field of pregnancy testing kits where an anti-hCG antibody is present on the support, and is carried complexed with hCG by the flow of urine onto an immobilised second antibody that permits visualisation.

In one preferred embodiment of the invention, is a solid support having a proximal and distal end, comprising:
a sample application zone in the vicinity of the proximal end,
   a reaction zone distal to the sample application zone, and
a detection zone distal to the reaction zone,
   whereby said support has a capillary property that directs a flow of fluid sample applied in the application zone in a direction from the proximal end to the distal end.

The reaction zone comprises one or more bands of CAIII probe conjugated to a detection agent (e.g. colloidal gold) which CAIII probe conjugate is disposed on the solid support such that it can migrate with the capillary flow of fluid i.e. it is not immobilised. The detection zone comprises one or more capture bands comprising a population of CAIII probe immobilised on the solid support.

When a sample is applied to the sample application zone, it migrates towards the reaction zone by capillary flow. Any CAIII present in the sample reacts with the CAIII probe conjugate, and the complex so formed is carried by capillary flow to the detection zone. The detection zone, having CAIII probe permanently immobilised thereon, captures and immobilises any complex, resulting in a localised concentration of conjugate that can be visualised.

The zones as described herein generally do not overlap. They may be adjacently arranged with an absence or presence of an intervening gap of solid support devoid of band. A band may be disposed on a solid support by any means, for example, absorbed, adsorbed, coated, covalently attached or dried, depending on whether the reagent is required to be mobilised or not.

Reference is made in the description below to the drawings which exemplify particular embodiments of the invention; they are not at all intended to be limiting. The skilled person may adapt the device and substituent components and features according to the common practices of the person skilled in the art.

FIGS. 1A and B shows a preferred embodiment of a test strip of the invention. The strip 1 includes a proximal end 2 and a distal end 3. A sample application zone 4 is provided in the proximal end 2, a reaction zone 5 is adjacent thereto and a detection zone 6 is in the vicinity of the distal end 3.

A sample may be deposited onto the solid support 7 at the application zone 4 to transfer by capillary action to the detection zone 6. A protective layer 8 that covers either or both the surfaces of the solid support 7, except for a region of the sample application zone 4 may be provided. Such protective layer protects the sample and chemical constituency of the strip from contamination and evaporation.

One or more absorbent pads 9 in capillary contact with the sample application zone 4 of the solid support 7 may absorb and release sample as necessary; such pad 9 is typically placed on the surface of the solid support 7 that is the same or opposing the sample application zone 4. In FIG. 1B, the absorbent pad 9 is part of the sample application zone 4.

One or more other absorbent pads 9′ in capillary may be placed in contact with the detection zone 6 of the solid support 7, distal to any capture bands 11, 14. These pads 9′ may absorb fluid that has passed through the solid support; such pad 9′ is typically placed on the surface of the solid support 7 that is the same or opposing the sample application zone 4.

The solid support 7 may made from any suitable material that has a capillary action property, and may have the same properties as described above. It should also be capable of supporting a substance (e.g. non-immobilised CAIII probe), which, when hydrated, can migrate across the solid support by a capillary action fluid flow.

The solid support 7 may also comprise a band of CAIII probe conjugate 10, located in the reaction zone 5, at a position distal to the sample application zone 4. Any CAIII in the sample is carried by capillary action towards this band 10, where it reacts with the permanently immobilised CAIII probe conjugate.

The CAIII probe conjugate may be associated with or attached to a detection agent to facilitate detection. Examples of lab detection agents include, but are not limited to, luminescent labels; colorimetric labels, such as dyes; fluorescent labels; or chemical labels, such as electroactive agents (e.g., ferrocyanide); enzymes; radioactive labels; or radiofrequency labels.

More commonly, the detection agent is a particle. Examples of particles useful in the practice of the invention include, but are not limited to, colloidal gold particles; colloidal sulphur particles; colloidal selenium particles; colloidal barium sulfate particles; colloidal iron sulfate particles; metal iodate particles; silver halide particles; silica particles; colloidal metal (hydrous) oxide particles; colloidal metal sulfide particles; colloidal lead selenide particles; colloidal cadmium selenide particles; colloidal metal phosphate particles; colloidal metal ferrite particles; any of the above-mentioned colloidal particles coated with organic or inorganic layers; protein or peptide molecules; liposomes; or organic polymer latex particles, such as polystyrene latex beads. Preferable particles are colloidal gold particles. The size of the particles may be related to porosity of the membrane strip: the particles are preferably sufficiently small to be transported along the membrane by capillary action of fluid.

Colloidal gold may be made by any conventional means, such as the methods outlined in G. Frens, 1973 Nature Physical Science, 241:20 (1973). Alternative methods may be described in U.S. Pat. Nos. 5,578,577, 5,141,850; 4,775,636; 4,853,335; 4,859,612; 5,079,172; 5,202,267; 5,514,602; 5,616,467; 5,681,775.

The selection of particle size may influence such factors as stability of bulk sol reagent and its conjugates, efficiency and completeness of release of particles from the test strip, speed and completeness of the reaction. Also, particle surface area may influence stearic hindrance between bound moieties.

The number of particles present in the CAIII probe conjugate may vary, depending on the size and composition of the particles, the composition of the solid support, and the level of sensitivity of the assay.

The solid support 7 further comprises one or more capture bands 11 in the detection zone 10. A capture band comprises a population of CAIII probe permanently immobilised thereon. The CAIII:CAIII probe conjugate complex formed in the reaction zone 5 migrates towards the detection zone 6 where said band 11 captures migrating complex, and concentrates it, allowing it to be visualised either by eye, or using a machine reader. The CAIII probe present in the reaction zone 5 and in the detection zone 6 may reaction to the same part of CAIII or may react to different parts of CAIII.

One or more controls bands 12 may be present on the solid support 7. For example, a non-immobilised peptide 12 might be present in the sample application zone 4, which peptide does not cross-react with any of bands of CAIII probes 13, 14. As the sample is applied, it migrates towards the reaction zone 5, where an anti-peptide antibody conjugate is disposed 13, and where a complex peptide-antibody complex is formed. Said complex migrates towards the detection zone 6, where a capture band 14 of anti-peptide antibody is immobilised on the solid support, and which concentrates said complex enabling visualisation. The control capture band 14 is located separately from the CAIII capture band 11, therefore, a positive reaction can be seen distinct from the detection reaction if the assay is working correctly.

A particular advantage of a control according to the invention is that they are internal controls—that is, the control against which the CAIII measurement results may be compared is present on the individual solid support. Therefore, the controls according to the invention may be used to correct for variability in the solid support, for example. Such correction would be impractical with external controls that are based, for example, on a statistical sampling of supports. Additionally, lot-to-lot, and run-to-run, variations between different supports may be minimized by use of control binding agents and control agents according to the invention. Furthermore, the effects of non-specific binding may be reduced. All of these corrections would be difficult to accomplish using external, off-support, controls.

During the assay, CAIII from the sample and the CAIII probe conjugate combine and concentrate on the solid support 7. This combination results in a concentration of compounds that may can be visualised above the background colour of the solid support 7. The compounds may be formed from a combination of above-mentioned compounds, including antibodies, detection agents, and other particles associated with the reaction and detection zones. Based on the particular assay being performed, the reaction and detection zones may be selectively implemented to achieve an appropriate dynamic range which may be linear or non-linear.

A solid support 7 for performing the assay may be housed within the cartridge 20 as shown, for example, in FIG. 2. The cartridge is preferably watertight against urine, except for one or more openings. The solid support 7 may be exposed through an opening 21 in the cartridge to provide an application zone 4 in proximal end 2, and another opening 22 to enable reading of detection zone 6 close to the distal end 3. Cartridge 20 may include a sensor code 23 for communicating with a reading device.

### Surface Plasmon Resonance

The presence and/or concentration of CAIII in a sample can be measured by surface plasmon resonance (SPR) using a chip having CAIII probe immobilized thereon. Surface plasmon resonance monitors the change in mass near the immobilised sensor. This change in mass is measured as resonance units versus time after injection or removal of the sample, and is measured using a Biacore Biosensor (Biacore AB). CAIII probe can be immobilised on a sensor chip (for example, research grade CM5 chip; Biacore AB) according to methods described by Salamon et al. (Salamon et al., 1996, Biophys J. 71: 283-294; Salamon et al., 2001, Biophys. J. 80: 1557-1567; Salamon et al., 1999, Trends Biochem. Sci. 24: 213-219, each of which is incorporated herein by reference). Conditions for CAIII binding to CAIII probe in an SPR assay can be fine-tuned by one of skill in the art using the conditions reported by Sarrio et al. (Sarrio et al., 2000, Mol. Cell. Biol. 20: 5164-5174, incorporated herein by reference) as a starting point. Binding reactions can be performed at different concentrations of immobilized CAIII probe, if necessary, to arrive at a concentration of CAIII in the sample. If a qualitative result is desired, controls and different concentrations may not be necessary. While CAIII probe is immobilised in the above, the skilled person may readily adapt the method so that the sample is the immobilised component.

### Fluorescence Resonance Energy Transfer

Another method of determining the concentration of CAIII in a sample is by using fluorescence resonance energy transfer (FRET). FRET is a quantum mechanical phenomenon that occurs between a fluorescence donor (D) and a fluorescence acceptor (A) in close proximity to each other (usually <100 angstroms of separation) if the emission spectrum of D overlaps with the excitation spectrum of A. The molecules to be tested, e.g., CAIII and CAIII probe, are labelled with a complementary pair of donor and acceptor fluorophores. While bound closely together by the CAIII: CAIII probe interaction, the fluorescence emitted upon excitation of the donor fluorophore will have a different wavelength than that emitted in response to that excitation wavelength when the CAIII and CAIII probe are not bound, providing for quantitation of bound versus unbound molecules by measurement of emission intensity at each wavelength. Donor fluorophores with which to label the sclerostin are well known in the art. Of particular interest are variants of the A. victoria GFP known as Cyan FP(CFP, Donor (D)) and Yellow FP (YFP, Acceptor(A)). As an example, the YFP variant can be made as a fusion protein with sclerostin. Vectors for the expression of GFP variants as fusions (Clontech) as well as fluorophore-labelled hCG mimetic compounds (Molecular Probes) are known in the art. Binding reactions can be performed at different CAIII probe concentrations, if necessary, to arrive at a concentration of CAIII. If a qualitative result is desired, controls and different concentrations may not be necessary.

### Bioluminescence Resonance Energy Transfer

Another detection system is bioluminescence resonance energy transfer (BRET), which uses light transfer between fusion proteins containing a bioluminescent luciferase and a fluorescent acceptor. In general, one molecule of the CAIII:CAIII probe complex is fused to a luciferase (e.g. Renilla luciferase (Rluc))—a donor which emits light in the wavelength of ˜395 nm in the presence of luciferase substrate (e.g. DeepBlueC). The other molecule of the pair is fused to an acceptor fluorescent protein that can absorb light from the donor, and emit light at a different wavelength. An example of a fluorescent protein is GFP (green fluorescent protein) which emits light at ˜510 nm. The addition of acceptor-fused candidate CAIII to the donor fused-CAIII probe will result in an energy transfer evidenced by, for example, an increase in acceptor fluorescence relative to a sample where an acceptor-fused CAIII does not bind. By measuring the interaction under a range of concentrations and conditions, if necessary, will provide the concentration of CAIII in a sample. If a qualitative result is desired, controls and different concentrations may not be necessary.

### Quenching

A variation on FRET uses fluorescence quenching to monitor molecular interactions. One molecule in the interacting pair can be labelled with a fluorophore, and the other with a molecule that quenches the fluorescence of the fluorophore when brought into close apposition with it. A change in fluorescence upon excitation is indicative of a change in the association of the molecules tagged with the fluorophore:quencher pair. Generally, an decrease in fluorescence of the labelled sclerostin is indicative that a candidate minetic bearing the quencher has been bound. Of course, a similar effect would arise when mimetic is fluorescently labelled and sclerostin bears the quencher. Binding reactions can be performed at different mimetic and/or sclerostin concentrations if necessary to arrive at a binding constant. For quenching assays, a 10% or greater (e.g., equal to or more than 20%, 30%, 40%, 50%, 60%) decrease in the intensity of fluorescent emission, indicates that the candidate hCG mimetic binds sclerostin. Control experiments using quench-labelled sclerostin and hCG can establish expected levels of quenching; a quenching observed with an hCG mimetic would be at least 10% (e.g., at least 20%, 30%, 40%, 50%, 60%, 70%, 80% or 90%) of the level observed with hCG.

In addition to the surface plasmon resonance and FRET methods, fluorescence polarization measurement is useful to quantitate binding. The fluorescence polarization value for a fluorescently-tagged molecule depends on the rotational correlation time or tumbling rate. Complexes, such as those formed by CAIII associating with a labeled CAIII probe, have higher polarization values than uncomplexed, labeled CAIII probe. Binding reactions can be performed at different CAIII probe concentrations if necessary to arrive at a concentration for CAIII in the sample. Control experiments using CAIII and CAIII probe can establish expected levels of polarization. If a qualitative result is desired, controls and different concentrations may not be necessary.

Any of the binding assays described can be used to determine the presence and/or concentration of CAIII in a urine sample. To do so, CAIII-probe is reacted with a sample, and the concentration of CAIII is measured as appropriate for the binding assay being used.

To validate and calibrate an assay, control reactions using different concentrations of standard CAIII and/or CAIII probe can be performed. Where solid phase assays are employed, after incubation, a washing step is performed to remove unbound CAIII. Bound, CAIII is measured as appropriate for the given label (e.g., scintillation counting, fluorescence, antibody-dye etc.). If a qualitative result is desired, controls and different concentrations may not be necessary. Of course, the roles of CAIII and CAIII probe may be switched; the skilled person may adapt the method so CAIII probe is applied to sample, at various concentrations of sample.

### CAIII Probe

A CAIII probe according to the invention is any substance that binds specifically to CAIII. Examples of a CAIII probe useful according to the present invention, includes, but is not limited to an antibody, a polypeptide, a peptide, a lipid, a carbohydrate, a nucleic acid, peptide-nucleic acid, small molecule, small organic molecule, or other drug candidate. A CAIII probe can be natural or synthetic compound, including, for example, synthetic small molecule, compound contained in extracts of animal, plant, bacterial or fungal cells, as well as conditioned medium from such cells. Alternatively, CAIII probe can be an engineered protein having binding sites for CAIII. According to an aspect of the invention, a CAIII probe binds specifically to CAIII with an affinity better than 10⁻⁶ M.

A suitable CAIII probe can be determined from its binding with a standard sample of CAIII. Methods for determining the binding between CAIII probe and CAIII are described above.

A CAIII probe useful according to the present invention may be an antibody or antigen-binding fragment thereof which specifically binds to CAIII. As used herein, the term antibody includes, but is not limited to, polyclonal antibodies, monoclonal antibodies, humanised or chimeric antibodies, engineered antibodies, and biologically functional antibody fragments (e.g. scFv, nanobodies, Fv, etc) sufficient for binding of the antibody fragment to the protein.

Such antibody may be commercially available antibody against CAIII, such as, for example, mouse monoclonal antibody clone 2CA-4 (Spectral).

According to one aspect of the invention, the CAIII probe is labelled with a tag that permits detection with another agent (e.g. with a probe binding partner). Such tags can be, for example, biotin, streptavidin, his-tag, myc tag, maltose, maltose binding protein or any other kind of tag known in the art that has a binding partner. Example of associations which can be utilised in the probe:binding partner arrangement may be any, and includes, for example biotin:streptavidin, his-tag:metal ion (e.g. Ni²⁺), maltose:maltose binding protein.

### B. Reagent Strip and Test Pad

In another embodiment, the invention provides a simple and accurate colorimetric reagent strip and method for measuring presence of CAIII in urine. More in particular, the present invention also relates to a device comprising a reagent strip. The present reagent strip comprises a solid support which is provided with at least one test pad for measuring the presence of CAIII in an urine sample. Said test pad preferably comprises a carrier matrix incorporating a reagent composition capable of interacting with CAIII to produce a measurable response, preferably a visually or instrumentally measurable response.

The reagent strip may be manufactured in any size and shape, but in general the reagent strip is longer than wide.

The solid support may be composed of any suitable material and is preferably made of firm or stiff material such as cellulose acetate, polyethylene terephthalate, polypropylene, polycarbonate or polystyrene.

In general, the carrier matrix is an absorbent material that allows the urine sample to move, in response to capillary forces, through the carrier matrix to contact the reagent composition and produce a detectable or measurable color transition. The carrier matrix can be any substance capable of incorporating the chemical reagents required to perform the assay of interest, as long as the carrier matrix is substantially inert with respect to the chemical reagents, and is porous or absorbent relative to the soluble components of the liquid test sample. The expression “carrier matrix” refers to either bibulous or nonbibulous matrices that are insoluble in water and other physiological fluids and maintain their structural integrity when exposed to water and other physiological fluids. Suitable bibulous matrices include filter paper, sponge materials, cellulose, wood, woven and nonwoven fabrics and the like. Nonbibulous matrices include glass fiber, polymeric films, and preformed or microporous membranes. Other suitable carrier matrices include hydrophilic inorganic powders, such as silica gel, alumina, diatomaceous earth and the like; argillaceous substances; cloth; hydrophilic natural polymeric materials, particularly cellulose material, like cellulosic beads, and especially fibercontaining papers such as filter paper or chromatographic paper; synthetic or modified naturally-occurring polymers, such as crosslinked gelatin, cellulose acetate, polyvinyl chloride, polyacrylamide, cellulose, polyvinyl alcohol, polysulfones, polyesters, polyacrylates, polyurethanes, crosslinked dextran, agarose, and other such crosslinked and noncrosslinked water-insoluble hydrophilic polymers. Hydrophobic and nonabsorptive substances are not suitable for use as the carrier matrix of the present invention. The carrier matrix can be of different chemical compositions or a mixture of chemical compositions. The matrix also can vary in regards to smoothness and roughness combined with hardness and softness. However, in every instance, the carrier matrix comprises a hydrophilic or absorptive material. The carrier matrix is most advantageously constructed from bibulous filter paper or nonbibulous polymeric films. A preferred carrier matrix is a hydrophilic, bibulous matrix, including cellulosic materials, such as paper, and preferably filter paper or a nonbibulous matrix, including polymeric films, such as a polyurethane or a crosslinked gelatin.

A reagent composition which produces a colorimetric change when reacted with CAIII in urine can be homogeneously incorporated into the carrier matrix, and the carrier matrix then holds the reagent composition homogeneously throughout the carrier matrix while maintaining carrier matrix penetrability by the predetermined component of the test sample.

Examples of suitable reagent compositions may include for instance a CAIII probe in case of an antibody-based technique, or pH buffer in case of enzymatic detection.

The reagent composition is preferably dried and stabilized onto a test pad adhered to at least one end of a solid support. The test pad onto which the reagent composition is absorbed and dried, is preferably made of a membrane material that shows minimal background color. Preferably, the test pad may be constructed of acid or base washed materials in order to minimize background color.

In another embodiment the reagent composition which is dried onto the reagent strip further comprises wetting agents to reduce brittleness of the test pad. Non-limiting examples of preferred wetting agents include TritonX-100, Bioterg, glycerol, 0 Tween, and the like.

The concentration of the reagent composition required on a dry pad is sufficient to allow discrimination in color development between 10 to 200 mg/L CAIII concentration. Preferably, the reagent strip contains about a sufficient amount of reagent composition, which can be determined by a skilled person. The reagent composition can be applied to the reagent strip by any method known in the art. For example, the carrier matrix from which the test pads are made may be dipped into a solution of the reagent composition and dried according to techniques known in the art.

A reagent strip according to the invention may be provided with multiple test pads to assay for more than one analyte in a urine sample. A reagent strip may be provided comprising a solid support provided with one or more test pads including test pads for measuring the presence of one or more analytes selected from the group comprising proteins, blood, leukocytes, nitrite, glucose, ketones, creatinine, albumin, bilirubin, urobilinogen and/or a pH test pad, and/or a test pad for measuring specific gravity.

A possible embodiment of a reagent strip 101 according to the invention is depicted diagrammatically in FIG. 14A-B. The strip 101 includes a proximal end 102 and a distal end 103. Various test pads 109, 109′, 109″ on which the reagent compositions are provided at the proximal end 102 on a solid support 107 of the reagent strip. The strip must be designed in such a way that it can be wetted with a sufficiently large amount of urine.

A reagent strip as defined herein is used as follows. Briefly, one or more test pad areas of the reagent strip of the invention is dipped into a urine sample or a small amount of urine sample is applied to the reagent strip onto the test pad area(s). A color development which can be analyzed visually or by reflectometry occurs on the reagent strip within a short time, usually within 0.5 to 10 minutes. The change in color of the reagent area on the test pad upon reacting with CAIII is preferably directly proportional to the concentration of CAIII in the patient sample. The color intensity that develops on the test pad may be determined visually or by a reflectance-based reader, for example. Color development at the test pad area(s) is compared to a reference color or colors to determine an estimate of the amount of CAIII present in the sample The color intensity that develops on the test pad is compared to at least one, and preferably at least two standard color shades that correspond to a range of CAIII concentration determined by application of a correction factor.

The reagent strip may further comprises an infrared dye, applied either to the support strip or incorporated into a test pad, which ensures proper alignment of the reagent strip in an apparatus having a detection system for the detectable or measurable response.

In another embodiment, the invention also relates to a test pad for measuring the presence of CAIII in a urine sample. Preferably said test pad comprises a carrier matrix incorporating a reagent composition capable of interacting with CAIII to produce a measurable response, preferably a visually or instrumentally measurable response. In another preferred embodiment the invention provides a test pad according as define herein for use in on a reagent strip, preferably on a reagent strip as defined herein.

### Diagnostic Kit

One embodiment of the present invention is a kit for diagnosing a condition related to dysfunction of the RPT in a subject.

Another embodiment of the present invention is a kit for monitoring the progress of a condition related to dysfunction of the RPT in a subject.

Another embodiment of the present invention is a kit for monitoring the effectiveness of treatment of a subject with an agent.

Yet another embodiment of the present invention is a kit for preventive screening of subjects for the presence of a condition related to dysfunction of the RPT in said subject.

Still another embodiment of the present invention is a kit for monitoring renal/kidney transplantation(s) in a subject. Monitoring is intended to refer to include “follow-up” of patients after kidney transplantation.

In one embodiment the invention relates to a diagnostic kit comprising a dipstick device as defined herein. In a preferred embodiment said kit comprises:
a solid support provided with means for determining the concentration and/or enzyme activity of CAIII in a urine sample,
an urine sample container and
optionally control standards comprising CAIII,

In a more preferred embodiment said means comprise at least one CAIII specific probe, and preferably a CAIII specific probe as defined herein. Said means for determining CAIII enzyme activity may comprise a pH buffer. Preferably the kit comprises a solid support whereby said CAIII probe or the means for determining CAIII enzyme activity is immobilised thereon.

More preferably a kit according to the invention comprises a solid support which has fluid capillary properties and comprises:
a distal (3) and proximal end (2),
a sample application zone (4) in the vicinity of the proximal end (2),
a reaction zone (5) distal to the sample application zone (4),
a detection zone (6) distal to the reaction zone (5),
where the reaction zone (5) is disposed with CAIII probe labelled with detection agent, that can migrate towards the distal end (3) in a flow of fluid by capillary action,
where the detection zone (6) comprises said immobilised CAIII probe that can capture CAIII.

In a preferred embodiment said solid support is housed in a cartridge (20) watertight against urine, having an opening (21) to provide access to the application zone (4) in proximal end (2), and another opening (22) to enable reading of detection zone (6) close to the distal end (3). Said cartridge (20) is preferably disposed with a sensor code (23) for communicating with a reading device.

Thus, a kit according to the invention may comprise one or more of the following components:
CAIII probe, preferably antibody directed against CAIII
solid support provided with or coated with CAIII probe,
control standards comprising CAIII,
a solid support having fluid capillary properties comprising:
   a distal and proximal end,
   a sample application zone in the vicinity of the proximal end,
   a reaction zone distal to the sample application zone,
   a detection zone distal to the reaction zone,
   where the reaction zone is disposed with CAIII probe labelled with detection agent, that can migrate towards the distal end in a flow of capillary action,
   where the detection zone comprises immobilised CAIII probe that can capture CAIII.
solid support having fluid capillary properties, housed in a cartridge as described herein, and
Urine sample container.

In another embodiment, the invention relates to a diagnostic kit comprising a reagent strip as defined herein. Preferably the invention relates to a diagnostic kit used to measure the presence of CAIIII in urine comprising at least one a reagent strip as defined herein, and an urine sample container. The diagnostic kit may optionally contain further constituents, such as, for example, standard solutions, a description of the method for using the reagent strip, or a color chart for visual evaluation.

### Applications

The present use of type III carbonic anhydrase as urinary marker find various applications in the medicinal field, including diagnostic, prophylactic as well as monitoring applications. Detection and quantification of CAIII in urine samples helps to identify patients with inherited or acquired, acute or chronic (follow-up) RPT dysfunction. In addiction, the non-invasive assay, which is based on the immediate measurement of urinary CAIII (e.g. using a dip stick or reagent strip as defined herein), could be useful in preventive medicine such as scholar medicine and occupational medicine for the industry, as well in curative medicine and in hospitals. Moreover the present simple and low-cost test might also facilitate the diagnosis and follow-up of patients with RPT dysfunction in the third world in view of high prevalence of RPT dysfunction caused by heavy metal intoxications. Finally, monitoring kidney safety in drug development needs new technologies and reliable assays such as the one proposed in the present invention.

More in particular in one aspect, the invention relates to the use of CAIII for the preparation of a diagnostic test for diagnosing a condition related to dysfunction of the RPT in a subject. The invention relates to CAIII for use in diagnosing a condition related to dysfunction of the RPT in a subject. In still another embodiment the invention relates to a method for diagnosing a condition related to dysfunction of the RPT in a subject

Early diagnosis of RPT dysfunction is essential for early and efficient treatment. The present invention provides a urinary biomarker which enables early and sensitive detection of RPT diseases. The terms “diseases related to a dysfunction of the renal proximal tubule (RPT)” or “RPT-related diseases” as used herein refers to diseases wherein dysfunction of the renal proximal tubule(s), as defined herein, plays a crucial detrimental role. Diseases related to a dysfunction of the RPT may include a pathology causing or a condition related to renal proximal tubule (RPT) dysfunction in a subject as enumerated above. The present invention provides a urinary biomarker which enables early and sensitive detection of a pathology causing or a condition related to renal proximal tubule (RPT) dysfunction in a subject. Therefore the present invention further relates to the use of CAIII for the preparation of a diagnostic test for diagnosing a pathology causing or a condition related to renal proximal tubule (RPT) dysfunction in a subject. The invention further relates to CAIII for use in diagnosing a pathology causing or a condition related to renal proximal tubule (RPT) dysfunction in a subject. In still another embodiment the invention relates to a method for diagnosing a pathology causing or a condition related to renal proximal tubule (RPT) dysfunction in a subject.

In another embodiment, the invention relates to the use of CAIII for the preparation of a test for preventive screening of subjects, such as school children or adults, for the presence of a pathology causing or a condition related to RPT in said subject.

In yet another embodiment, the invention relates to the use of CAIII for the preparation of a test for monitoring (following up) kidney transplantation in a subject. The invention further relates to CAIII for use in monitoring (following up) kidney transplantation in a subject. In still another embodiment the invention relates to a method for monitoring (following up) kidney transplantation in a subject. For instance in case of transplant rejection, RPT dysfunction may occur, and can be detected by urine analyses for CAIII.

### Examples

In this study, we have used Clcn5 knockout mice, as a well-defined model of renal Fanconi syndrome, in order to investigate the metabolic consequences and adaptative mechanisms to PT dysfunction. The Clcn5^{Y/-} kidneys were characterized by higher cell proliferation and oxidative stress, and a 4-fold upregulation of CAIII—which was previously unknown in the kidney. CAIII was exclusively distributed in the cytosol of scattered PT cells. These findings were confirmed in kidney samples from a patient with Dent's disease, and extended to other mouse models of PT dysfunction and PT cell lines exposed to oxidant conditions. As a whole, these results show that generalized PT dysfunction is associated with increased cell proliferation, dedifferentiation and oxidative stress. The kidney-specific upregulation of CAIII, which is an early mesodermal marker, may play a role in PT cell defense against oxidative damage.

### 1. Materials and Methods

### Mouse Models.

Experiments were conducted on 12-week-old and 1-year-old Clcn5 wild-type (Y/+) and KO (Y/−) mice. The Clcn5^{Y/-} mice, generated by targeted deletion of the exon VI of Clcn5, have been extensively characterized and mimic the phenotype of human Dent's disease (Wang, S. S. et al. 2000 Hum. Mol. Genet. 9: 2937-2945). Animals were kept in metabolic cages for 24 h with ad libitum access to food and drinking water, and urine was collected on ice-cold Complete™ protease inhibitors (Roche, Vilvoorde, Belgium). An aliquot was used to measure the levels of creatinine by standard methods (Eastman Kodak Company, Rochester, N.Y., USA). Kidneys and urine from two additional mouse models of human renal Fanconi syndrome of variable severity, i.e. 15-week-old megalin KO mice (Willnow, T. E. et al. 1996 Proc. Natl. Acad. Sci. U.S.A. 93: 8460-8464), and 24-week-old Ctns KO mice (Cherqui, S. et al. 2002, Mol. Cell. Biol. 22: 7622-7632) were also used. The megalin KO mice exhibit a specific defect in PT endocytic apparatus resulting in impaired uptake of filtered LMW proteins, without significant alteration of glucose, electrolyte and amino acid transports (Leheste, J. R et al. 1999. Am. J. Pathol. 155: 1361-1370). The Ctns KO mice present no signs of proximal tubulopathy despite the severe PT defects observed in children with infantile cystinosis, which suggests alternative rescue pathways in mouse (Cherqui, S. et al., 2002, Mol. Cell. Biol. 22: 7622-7632). All samples were obtained in accordance with NIH guidelines for the care and use of laboratory animals, and with the approval of the Committee for Animal Rights of the UCL Medical School.

### Human Samples.

Frozen and formalin-fixed kidney samples (outer cortex) were obtained at time of bilateral nephrectomy in an end-stage patient with Dent's disease. The clinical features of this patient, who harbours the missense mutation Gly506Glu in CLCN5, were reported previously (Lloyd, S. E. et al. 1996. Nature. 379: 445-449). Cortical samples from Four end-stage kidneys (chronic interstitial nephritis) removed during renal transplantation were used as controls. These samples were snap-frozen in liquid nitrogen and stored at −80° C., or routinely fixed in 4% formaldehyde. We obtained urine samples (second morning miction) from three unrelated patients with Dent's disease harboring nonsense mutations (S203X, R637X, and R648X) in CLCN5 and from their asymptomatic carrier mothers. All patients (aged 7, 15, and 13 years, respectively) had LMW proteinuria and hypercalciuria but no renal failure.

The use of these samples has been approved by the Ethical Review Board of the UCL Medical School.

### Proximal Tubule Cell Lines.

The human kidney (HK2) and Opossum kidney (OK) cell lines are established models of PT cells (Ryan, M. J. et al. Kidney Int. 45: 48-57). The HK-2 cell line was obtained from ATCC (Teddington, UK) and grown on keratinocyte-serum free medium (GIBCO-BRL 17005-042, Invitrogen) with 5 ng/ml recombinant epidermal growth factor, 50 pg/ml bovine pituitary extract, 50 U/ml penicillin, and 50 pg/ml streptomycin, at 37° C. in a 95% air/5% CO₂ incubator. The OK cells were grown on DMEM-F12 medium (GIBCO-BRL 31330-038, Invitrogen), with 10 U/ml penicillin, 10 μg/ml streptomycin, and foetal bovine serum 10%, at 37° C. in a 95% air/5% CO₂ incubator. When the cultures reached confluence, subcultures were prepared using a 0.02% EDTA-0.05% trypsin solution (Invitrogen). After 24 h-deprivation of serum, HK-2 cells (passage 12) and OK cells (passage 111) were treated with H₂O₂ (1 mM or 0.3 mM, respectively). At various times post H₂O₂-treatment, cells were trypsinized, washed twice in cold PBS, and centrifuged at 300 g for 5 min. The pellet was harvested at −80° C. before mRNA and protein extraction.

RNA Extraction and Double Strand cDNA Synthesis.

Total RNA was extracted from frozen samples (human and mouse kidneys, cell lysates) using Trizol reagent (Invitrogen, Merelbeke, Belgium). The concentration of each RNA sample was obtained from optical densitometry (260 nm) measurements and RNA quality was confirmed using agarose gel electrophoresis. For AFLP, poly(A)+ RNA were prepared from 75 pg of total RNA using Dynabeads Oligo(dT)₂₅ (Invitrogen). First strand cDNA was synthesized from 500 ng of Poly(A)+ RNA using Superscript II RNase H⁻ Reverse Transcriptase (Invitrogen) in a total volume of 20 μl at 37° C. for 50 min. Double strand cDNA was synthesised in the same vial using T4 DNA Polymerase and purified using QIAquick Extraction Kit (Qiagen, Venlo, The Netherlands).

### AFLP Reactions.

The AFLP protocol was performed as previously described (42). cDNA samples were digested with EcoRI and Msel (Fermentas, Vilnius, Lithuania) for 2 h at 37° C. Restriction fragments were next ligated to EcoRI and Msel double strand adapters (Table 2) for 2 h at 20° C.

TABLE 2
Nucleotide sequence of adapters and primers
used for AFLP reactions

| Name | Sequence |
|---|---|
| Ad1-Eco | 5′ CTCGTAGACTGCGTACC 3′ |
| Ad2-Eco | 5′ AATTGGTACGCAGTCTAC 3′ |
| Ad1-Mse | 5′ GACGATGAGTCCTGAG 3′ |
| Ad2-Mse | 5′ TACTCAGGACTCAT 3′ |
| Eco-P0 | 5′ GACTGCGTACCAATTC 3′ |
| Mse-P0 | 5′ GATGAGTCCTGAGTAA 3′ |
| Eco-PAA | 5′ GACTGCGTACCAATTCAA 3′ |
| Eco-PAC | 5′ GACTGCGTACCAATTCAC 3′ |
| Eco-PAG | 5′ GACTGCGTACCAATTCAG 3′ |
| Eco-PAT | 5′ GACTGCGTACCAATTCAT 3′ |
| Mse-PAA | 5′ GATGAGTCCTGAGTAAAA 3′ |
| Mse-PAC | 5′ GATGAGTCCTGAGTAAAC 3′ |
| Mse-PAT | 5′ GATGAGTCCTGAGTAAAT 3′ |

The restriction fragments with ligated adapters were diluted (10×) with TE buffer (100 mM Tris-HCl, 10 mM EDTA, pH 8.0), and used as a template for the pre-amplification reaction which was performed for 20 cycles (94° C., 30 sec; 56° C., 1 min; 72° C., 1 min) using Eco-P0 and Mse-P0 primers. The product was diluted (10×) with TE buffer and 5 μl were used for selective amplification, as follows: 33 cycles including 9 touchdown cycles comprising a decrease of the annealing temperature from 65° C. to 56° C., which was maintained for 24 cycles. Twelve primer combinations were used for selective amplification: Eco-PAA and Mse-PAA or Mse-PAC or Mse-PAT, Eco-PAC and Mse-PAA or Mse-PAC or Mse-PAT, Eco-PAG and Mse-PAA or Mse-PAC or Mse-PAT, Eco-PAT and Mse-PAA or Mse-PAC or Mse-PAT. All amplification reactions were performed in the iCycler thermal cycler (Bio-Rad, Nazareth, Belgium). Selective amplification products were denatured at 95° C. for 3 min and separated on sequencing gels (6% polyacrylamide, 6 M urea) that were then dried and exposed to Kodak BioMax film (Amersham Biosciences, Buckinghamshire, UK) overnight at −80° C. The bands of interest were removed from the gel and soaked in water. AFLP fragments were recovered by PCR under the same conditions as used for the selective amplification. Reamplified cDNAs were visualised on a 1.5% (w/v) agarose gel, subcloned into pGEM-T easy vector (Promega, Leiden, The Netherlands) and sequenced (Genome Express, Meylan, France). Each reamplified AFLP fragment was compared against all sequences in the non-redundant databases using BLAST sequence alignment program: http://www.ncbi.nlm.nih.qov/BLAST/ (Altschul, S. F. et al. J. Mol. Biol. 215: 403-410).

### Real-Time RT-PCR.

Total RNA was treated with DNase I (Invitrogen) and reverse-transcribed into cDNA using SuperScript III RNase H⁻ Reverse Transcriptase (Invitrogen). Changes in mRNA expression levels were determined by real-time RT-PCR (iCycler IQ System, Bio-Rad) using SYBR Green I (Invitrogen) detection of single PCR product accumulation. Specific primers were designed using Beacon Primer Designer 2.0 (Premier Biosoft International, Palo Alto, Calif.) and are listed in Table 3.

TABLE 3

Primers used for real-time RT-PCR

| | | | Length | |
|---|---|---|---|---|
| Primer pair | Forward | Reverse | (bp) | Efficiency |
| Mouse | | | | |
| | 5′ CTTGAAGCACTGCATTCCAT 3′ | 5′ CACGATCCAGGTCACA CATT 3′ | 153 | 1.03 ± 0.09 |
| Car2 | | | | |
| | 5′ CTTGATGC CCTGGACAAAAT 3′ | 5′ GAGCCGTGGTAGGTCCAATA 3′ | 110 | 1.04 ± 0.11 |
| Car3 | | | | |
| PCNA | 5′ TTGGAATCCCAGAACAGGAG 3′ | 5′ ATTGCCAAGCTCTCCACTTG 3′ | 155 | 1.02 ± 0.20 |
| | 5′ TGCAAAGGTAGAGGCTCCAT 3′ | 5′ CAGGTAGGCCAGAGCAAGT 3′ | 152 | 1.03 ± 0.17 |
| Ki67 | | | | |
| osteopontin | 5′ TCCAATCGTCCCTACAGTCG 3′ | 5′ CGCTCTTCATGTGAGAGGTG 3′ | 146 | 0.98 ± 0.08 |
| catalase | 5′ CATGGTCTGGGACTTCTGGA 3′ | 5′ GACTGCCTCTCCATCTGCAT 3′ | 151 | 0.97 ± 0.27 |
| Type I SOD | 5′ GGGTTCCACGTCCATCAGTA 3′ | 5′ CAGTCACATTGCCCAGGTCT 3′ | 136 | 1.10 ± 0.09 |
| thioredoxin | 5′ TGATCAAGCCCTTCTTCCAT 3′ | 5′ CCCACCTTTTGACCCTTTTT 3′ | 151 | 1.00 ± 0.20 |
| | 5′ TGCACCACCAACTGCTTAGC 3′ | 5′ GGATGCAGGGATGATGTTCT 3′ | 176 | 1.04 ± 0.03 |
| gapdh | | | | |
| Hprt1 | 5′ ACATTGTGGCCCTCTGTGTG 3′ | 5′ TTATGTCCCCCGTTGACTGA 3′ | 162 | 0.99 ± 0.01 |
| Human | | | | |
| | 5′ CCCTGGATGGCACTTACAG 3′ | 5′ CAGCTTTCCCAAAATCCCCA CATT 3′ | 153 | 1.01 ± 0.10 |
| Car2 | | | | |
| | 5′ GCCGGGACTACTGGACCTA 3′ | 5′ CGTTCTCAGCACTGGAGAG 3′ | 144 | 0.97 ± 0.11 |
| Car3 | | | | |
| | 5′ ACGTCTCTTTGGTGCAGCTC 3′ | 5′ GCGTTATCTTCGGCCCTTAG 3′ | 157 | 0.98 ± 0.30 |
| PCNA | | | | |
| osteopontin | 5′ ATGGCCGAGGTGATAGTGTG 3′ | 5′ GATGGCCTTGTATGCACCAT 3′ | 146 | 1.10 ± 0.30 |
| catalase | 5′ TGGCTCATTTTGACCGAGAG 3′ | 5′ GCGATGGGAGTCTTCTTTCC 3′ | 148 | 0.95 ± 0.26 |
| thioredoxin | 5′ TCAGCCACGTGGTGTGGG 3′ | 5′ TGGAATGTTGGCATGCATTTGA 3′ | 152 | 1.20 ± 0.30 |
| GAPDH | 5′ GGGGCTCTCCAGAACATCAT 3′ | 5′ TCTAGACGGCAGGTCAGGT 3′ | 149 | 0.97 ± 0.12 |

The PCR products were size-fractionated on 1.5% agarose gel, stained with ethidium bromide, purified by QIAquick Gel Extraction Kit (Qiagen) and sequenced by Genome Express. Real-time RT-PCR analyses were performed in duplicate with 200 nM of both forward and reverse primers in a final volume of 25 μl using 1 Unit of Platinum Taq DNA Polymerase, 6 mM MgSO₄, 400 μM dNTP and SYBR Green I diluted 1/100,000. The PCR mix contained 10 nM fluorescein for initial well-to-well fluorescence normalization. PCR conditions were as follows: 94° C. for 3 min, 40 cycles of 30 sec at 95° C., 15 sec at 61° C. and 1 min at 72° C. The melting temperature of the PCR product was checked at the end of each PCR by recording SYBR Green fluorescence increase upon slow renaturing DNA. For each assay, standard curves were prepared by serial 4-fold dilutions of WT mouse kidney cDNA. The efficiencies of the amplifications with each primer set were calculated from the slope of the standard curve [efficiency=(10^{-1/slope})−1] and were close to 1 (Table 3). The relative changes in Target mRNA/GAPDH (or HPRT1) mRNA ratio between Clcn5^{Y/+} and Clcn5^{Y/-} samples were determined by using the formula: Efficiency ^{ΔΔCt.}

### Laser Capture Microdissection (LCM).

Frozen sections (7 μm) of Clcn5^{Y/-} and Clcn5^{Y/-} kidneys were mounted on appropriate coated slides (PALM MembraneSlides, P.A.L.M. Microlaser Technologies AG, Bernried, Germany), washed in H₂O for 1 min, dehydrated in a stepwise manner with 70%, 95% and 100% ethanol for 30 sec each, and finally dried in xylene for 5 min. Samples of ˜20,000 μm² were selectively microdissected from cortex and medulla regions using PALM Microsystem (Leica, Wetzlar, Germany) following manufacturer's recommendations. The microdissected samples were transferred to an eppendorf coated with PCR oil (Sigma M5904), pooled (total area of ˜140,000 μm² from each region), incubated with 60 μl of RNA lysis buffer (Ambion, Huntington, The United Kindgdom), and further processed for RNA extraction and quantification.

### Antibodies.

Mouse monoclonal antibodies against CAIII (Spectral, Toronto, Canada) (Azzazy, H. M., P. J. Cummings, D. R. Ambrozak, R. H. Christenson. 1998. 17: 553-558); V-ATPase E1 subunit (a gift of Dr. S. Gluck, University of California, San Francisco, Calif., USA); proliferative cell nuclear antigen (PCNA, clone PC-10, Dako, Heverlee, Belgium); and β-actin (Sigma, St-Louis, Mo.); rat monoclonal against Ki67 antigen (clone TEC-3, Dako); rabbit polyclonal antibodies against CAIII (Nishita, T., et al. Am. J. Vet. Res. 63: 229-235), vitamin D-binding protein (DBP) (Dako) and aquaporin-1 (Chemicon, Temecula, Calif.); and sheep polyclonal antibodies against CAII (Serotec, Oxford, UK) were used.

The specificity of anti-CAIII antibodies has been documented against purified CAI and CAII (Nishita, T., et al. Am. J. Vet. Res. 63: 229-235) and in reference tissue samples, including Car3 KO kidneys (FIG. 5), red blood cell ghosts, and epididymis (FIG. 12).

### Protein Extraction and Immunoblotting.

Cytosolic proteins were extracted from kidney samples as previously described (Wang, S. S., O. Devuyst, P. J. Courtoy, X. T. Wang, H. Wang, Y. Wang, R. V. Thakker, S. Guggino, W. B. Guggino. 2000. Hum. Mol. Genet. 9: 2937-2945). Briefly, tissues were homogenized in ice-cold buffer (0.25 M sucrose, 20 mM imidazole, pH 7.4, 1 mM EDTA) containing Complete™ protease inhibitors (Roche). A low-speed “nuclear” fraction was pelleted from the homogenate by centrifugation at 1000 g for 10 min and extracted twice by resuspension sedimentation. The resulting supernatant was centrifuged at 100,000 g for 60 min at 4° C. in a 50Ti fixed-angle rotor (Beckman, Palo Alto, Calif.). The supernatant was considered as the cytosolic fraction, and the high-speed pellet as the membrane compartment. To obtain cell lysates, the HK-2 and OK cells were harvested by trypsinization, and centrifuged twice at 1000×g for 10 min. After discarding the supernatant, the pellet was solubilized in ice-cold lysis buffer containing Complete MiniR (Roche), briefly sonicated (Branson Sonifier 250, 2 pulses at 40% intensity), and then centrifuged at 16,000×g for one minute at 4° C. Protein concentrations were determined using bicinchoninic acid protein assay (Pierce, Aalst, Belgium). Tissue and urine samples were thawed on ice, normalized for protein or creatinine levels, diluted in Laemmli buffer, separated through SDS-PAGE (10×7 cm, 14% gels) in reducing conditions, and transferred onto nitrocellulose membrane (Bio-Rad). After blocking, membranes were incubated overnight at 4° C. with primary antibodies, rinsed and incubated for 1 h at room temperature with the appropriate secondary peroxidase-labelled antibody (Dako). The immunoreactive bands were detected using enhanced chemiluminescence (Amersham Biosciences). Normalization for β-actin was obtained after stripping the blots. Specificity of the immunoblot was determined by incubation with non-immune rabbit or mouse IgG (Vector Laboratories, Brussels, Belgium). Densitometry analysis was performed with a Canon CanoScan8000F using the NIH Image V1.60 software. All immunoblots were performed in duplicate.

### Immunostaining.

Kidney samples were fixed for 6 h at 4° C. in 4% paraformaldehyde (Boehringer Ingelheim, Heidelberg, Germany) in 0.1M phosphate buffer, pH 7.4, prior to embedding in paraffin. Six-μm thick sections were rehydrated and incubated for 30 min with 0.3% hydrogen peroxide to block endogenous peroxidases. After incubation with PBS 10% normal goat serum for 20 min, sections were incubated for 45 min with primary antibodies in PBS 2% BSA. After washes, sections were incubated with appropriate biotinylated secondary antibodies (Vector Laboratories), washed and incubated for 45 min with the avidin-biotin peroxydase complex (Vectastain Elite, Vector Laboratories) and aminoethylcarbazole. Control experiments included incubation (i) in the absence of primary antibody, (ii) with non-immune rabbit or mouse IgG (Vector Laboratories). Counting of PCNA-, Ki67-, and CAIII-positive PT cells was blindly performed on 5 different cortex areas in 4 pairs of Clcn5^{Y/-} vs. Clcn5^{Y/+} kidneys.

Apoptosis assay. Apoptotic cells were detected in kidneys using the terminal deoxynucleotidyl transferase-mediated deoxyuridine triphosphate nick end labeling (TUNEL) method (Cell Death detection kit, Roche). Sections were pre-treated with 20 μg/ml proteinase K for 20 min. Positive control sections were first treated with 100 μg/ml DNAse I for 10 min at room temperature, whereas omission of transferase was regarded as negative control.

### Detection of Superoxide Anion (O₂⁻) Generation.

The in situ production of O₂⁻ in kidney sections was assessed using the hydroethidine (HE) fluorescence method (Piech, A., C. Dessy, X. Havaux, O. Feron, J. L. Balligand. 2003. Cardiovasc. Res. 57: 456-467). HE is freely permeable to cells, and is oxidized by O₂⁻ to red fluorescent ethidium bromide (EB). After excitation at 480 nm, EB emits light at a wavelength of 610 nm. After embedding in Tissue Tek OCT compound (Sakura Finetek, Zoeterwoude, The Netherlands), kidneys were snap-frozen in pre-cooled isopentane, cut into 5-μm-thick cryosections, and stored at −80° C. The 5 mM stabilized HE solution in DMSO (Invitrogen) was diluted to 2×10⁻⁶ M in water before use. The tissue sections were incubated with 50 μl of HE solution at 37° C. for 30 min in a light-protected and humidified chamber. Red fluorescence from HE-treated samples was measured during 5 ms using the software KS400 (Zeiss, Zaventem, Belgium) through a Zeiss Axiovert S100 microscope equipped with an Axiocam camera.

### Immunogold Labelling.

Kidneys were fixed by retrograde perfusion through the aorta with 2% formaldehyde in 0.1M sodium cacodylate buffer, pH 7.2. Tissues were trimmed into small blocks, further fixed by immersion for 1 h in 1% formaldehyde, infiltrated with 2.3 M sucrose for 30 min and frozen in liquid nitrogen. For electron microscopy (EM), 70-90 nm sections were obtained at −100° C. with an FCS Reichert Ultracut S cryoultramicrotome as previously described (Christensen, E. I. 1995. Eur. J. Cell. Biol. 66: 349-364). For EM immunolabeling the sections were incubated with rabbit anti-CAIII at 4° C. overnight followed by incubation for 1 hour with 10 nM goat anti-rabbit gold particles (BioCell, Cardiff, UK). The cryosections were embedded in methylcellulose containing 0.3% uranyl acetate and studied in a Philips CM100 electron microscope. Control sections were incubated with secondary antibody alone or with non-specific rabbit serum.

### Statistics.

Results are expressed as means±SD. Comparisons between groups were made by Student unpaired t-tests. The significance level was set at p<0.05.

### 2. Results

### 2.1. Metabolic Outcomes of CIC-5 Inactivation in Mouse Kidney

Real-time RT-PCR analyses showed a significant increase in differentiation markers, such as PCNA, Ki67, cyclin E and osteopontin, in the kidneys of Clcn5^{Y/-} mice, taken as a paradigm of renal Fanconi syndrome (FIG. 3). Furthermore, the increased mRNA expression of distinct reactive oxygen species (ROS) scavengers, such as type I superoxide dismutase (SOD) and thioredoxin, indicated an increased solicitation of cell oxidative defenses in Clcn5^{Y/-} kidneys in comparison to controls (FIG. 3). These results, obtained in 12-week-old mice, were also observed in 1-year-old Clcn5^{Y/-} mice (data not shown). Immunohistochemistry detected a significantly higher number of PCNA- and Ki67-positive cells in CIC-5 deficient kidneys (˜1.8% of PT cells) vs. controls (˜0.5% of PT cells) (FIG. 4). Comparative measurement of ethidium fluorescence in kidney sections revealed that the lack of CIC-5 was associated with a major increase in the production of superoxide O₂⁻ anion in PT cells (FIG. 4). These experiments showed that the PT dysfunction associated with inactivation of CIC-5 is reflected by a significant increase in cell proliferation and a major oxidative stress that solicitates ROS scavengers.

### 2.2. Comparison of Clcn5^{Y/+} and Clcn5^{Y/-} Renal Transcriptomes

We next sought to identify differentially expressed genes possibly involved in adaptative mechanisms against PT dysfunction using the AFLP procedure on 12-week-old Clcn5^{Y/-} and Clcn5^{Y/-} kidneys (n=4 pairs). Using one third of the possible AFLP primer combinations (see Materials and Methods), a total of 10 cDNA bands were reproducibly identified as differentially expressed in Clcn5^{Y/-} versus Clcn5^{Y/+} kidneys. One of these bands, significantly upregulated in Clcn5^{Y/-} samples, was identified as a transcript of Car3 (GenBank accession number: M27796), encoding Type III carbonic anhydrase (CAIII). The other cDNA bands, which were differentially expressed in Clcn5^{Y/-} vs. control kidneys, corresponded to unidentified mouse contigs. Quantitative real-time RT-PCR was used to validate these results, by comparing the mRNA expression of CAIII and CAII, the most abundant CA isoform in the kidney, in Clcn5^{Y/-} vs. Clcn5^{Y/+} kidneys (FIG. 5, panel A). As expected, CAIII mRNA expression was ˜5-fold lower than CAII in wild-type kidneys. However, the CAIII transcript was significantly upregulated in CIC-5 deficient kidneys (Ratio: 553%±48 of Clcn5^{Y/+} level, n=6), whereas CAII mRNA expression remained unchanged (94%±8 of Clcn5^{Y/+} level). These results were confirmed using laser capture microdissected cortex samples (data not shown). A similar, 5-fold induction of CAIII expression was also observed in the kidneys from 1-year-old Clcn5^{Y/-} mice in comparison to age-matched controls (data not shown). The upregulation of CAIII mRNA associated with the loss of CIC-5 was specific to the kidney, as CAIII mRNA expression levels in liver, skeletal muscle (vastus lateralis) and lung from Clcn5^{Y/-} mice were unchanged (FIG. 5, panel B).

These data demonstrate the usefulness of the AFLP procedure coupled with real-time RT-PCR to compare the transcriptome of distinct groups of mice. Using this approach, we evidenced a significant and kidney-specific induction of Car3 mRNA expression in mice lacking CIC-5.

### 2.3. Expression of CAIII in Clcn5^{Y/+} and Clcn5^{Y/-} Kidney

In order to investigate the upregulation of CAIII at protein level, we first validated the specificity of our antibodies raised against the closely related CAII and CAIII isoforms. Affinity-purified anti-CAIII antibodies, with no cross-reactivity for purified CAI and CAII (Nishita T, et al. Am J Vet Res 2002; 63: 229-235), were used to further investigate CAIII expression in the kidney. These antibodies did not detect any specific signal with Car3 KO kidney samples, whereas anti-CAII antibodies recognized the appropriate isoform in all samples (FIG. 5 panel F). The distinction between CAII and CAIII isoforms was facilitated by their different electrophoretic mobility in 14% SDS-PAGE, with CAIII showing a slightly faster migration pattern (˜27 kD) than CAII (˜29 kD) (FIG. 5 panel C). There was no cross-reactivity between the two isozymes, characterized by a slightly distinct molecular weight after SDS-PAGE migration (FIG. 5, panel C). Using these antibodies, immunoblotting analyses confirmed the consistent, 4-fold upregulation of CAIII in 12-week-old CIC-5 deficient kidneys, contrasting with the lack of changes in CAII expression (FIG. 5, panels D-E). These data were confirmed by using a commercial mouse monoclonal antibody directed against CAIII (data not shown).

Of note, immunoblotting analyses also detected a specific excretion of CAIII in the urine of the Clcn5^{Y/-} whereas it was not detected in Clcn5^{Y/+} or any wild-type mouse tested (FIG. 6 panel A). Immunoblotting analyses detected a specific excretion of CAIII in urine samples from Clcn5^{Y/-} mice (FIG. 6 panel A), and in the urine of three unrelated patients with Dent's disease (FIG. 6 panel B). It must be noted that CAIII was detected in simple, non-centrifuged urine samples, and that variable levels of CAII could also be detected in such samples, irrespective of the genotype and CAIII levels (data not shown). Thus, CAIII expression is significantly and specifically increased in CIC-5-deficient kidneys, and CAIII is detected in the urine of mice and patients lacking CIC-5.

These results support the data obtained by real-time RT-PCR, and further demonstrate that CAIII expression is significantly and specifically increased at both mRNA and protein levels in CIC-5 deficient kidneys. The detection of CAIII in Clcn5^{Y/-} urine also indicates that it represents a novel biomarker of PT dysfunction.

### 2.4. Cellular and Subcellular Distribution of CAIII in Mouse Kidney

In normal kidney, a weak immunoreactive signal for CAIII was observed in some tubules located in the outer cortex (FIG. 7, panel A). At higher magnification, the staining pattern was restricted to a subset of cells lining the PT, identified by their apical reactivity for the E1 subunit of the V-ATPase (panels C-D). Of note, CAIII was detected only in PT cells, and was not observed in any other cell types including the α-type IC, which express the V-ATPase at their apical membrane (panels C-D, arrowheads). In CIC-5 deficient kidney, the total number of CAIII-positive PT cells in the outer cortex was increased ˜4-fold (17.1% vs. 4.2% of Clcn5^{Y/-} PT cells) (panel B). In addition, immunoreactive signal for CAIII was detected in PT of the inner cortex. No signal was detected with non-immune rabbit IgG (panel E). The number of PT cells undergoing apoptosis established by the TUNEL reaction was similar in Clcn5^{Y/-} and Clcn5^{Y/-} kidneys (data not shown).

Subcellular fractionation of mouse kidneys demonstrated that CAIII was predominantly located in the cytosol, with residual distribution in membrane and nuclear fractions, as reported previously in adipocytes and hepatocytes (Tweedie, S., Y. Edwards. 1989. Biochem. Genet. 27: 17-30). Immunogold analyses showed that in normal kidney cortex, CAIII distribution was mainly cytosolic, also including the apical brush border microvilli (FIG. 8, panels A and D). Nuclei were labelled (FIG. 8, panels C and F), and a possible endosomal labelling could not be excluded (FIG. 8, panel B). No significant signal was noticed in mitochondria (FIG. 8, panel E). In Clcn5^{Y/-} samples, CAIII labelling appeared stronger than in Clcn5^{Y/-} kidneys, with a similar distribution (FIG. 8, panels F vs. C).

Further immunogold analyses showed that in normal kidney cortex, CAIII distribution was mainly cytosolic, also including the apical brush border microvilli (FIG. 11, panels A and D). Nuclei were labelled (FIG. 11, panels C and F), and a possible endosomal labelling could not be excluded (FIG. 11, panel A and D). No significant signal was noticed in mitochondria. In Clcn5^{Y/-} samples, CAIII labelling appeared stronger than in Clcn5^{Y/-} kidneys, with a similar distribution (FIG. 11, panels A-C vs. D-F). The predominant cytosolic distribution of CAIII, with residual distribution in membrane and nuclear fractions, was confirmed by subcellular fractionation of mouse kidneys.

Altogether these data demonstrate that the CAIII isozyme is present in mouse kidney, with a segmental distribution including the cytosol, the nucleus and the brush border of a subset of cells lining the PT in the outer cortex. The loss of the Cl⁻ transporter CIC-5 causes a significant increase of CAIII expression in PT cells of both outer and inner cortices, with a similar subcellular localization.

### 2.5. Upregulation of CAIII in Human Dent's Disease Kidney

The data obtained in the Clcn5^{Y/-} mouse model were assessed in kidney samples from one patient with renal Fanconi syndrome due to the inactivating Gly506Glu mutation of CLCN5 responsible for Dent's disease (Lloyd, S. E., 1996. Nature. 379: 445-449). There was a ˜5-fold upregulation of CAIII at both mRNA and protein levels in kidney samples with Dent's disease in comparison to 4 end-stage kidney samples taken as controls (FIG. 9, panels A and C). In addition, real-time RT-PCR studies showed that the functional loss of CIC-5 was associated with an induction of PCNA and thioredoxin expression, reflecting a higher cell proliferation and oxidative stress, respectively (FIG. 9, panel B). Despite tissue damage due to the end-stage renal disease, the expression of CAIII could be located in PT cells, identified by co-staining with the water channel aquaporin-1 (FIG. 9, panel D). These results demonstrate that the functional loss of CIC-5 in human kidney is associated with proliferation, protection against oxidative damage and induction of CAIII in PT cells.

Type II CA was also apparently upregulated in Dent kidney samples, potentially related to metabolic acidosis. The CAII induction was not observed in mice lacking CIC-5 (FIG. 5), which have no renal failure. Although restricted to a single human kidney, these results correlate with the detection of CAIII in the urine of 3 other patients with Dent's disease and normal renal function (FIG. 6B). They support the hypothesis that the loss of CIC-5 in the kidney is associated with proliferation, protection against oxidative damage and induction of CAIII in PT cells.

2.6. Expression of CAIII mRNA in Distinct Mouse Models of PT Dysfunction

As demonstrated above, the severe PT dysfunction caused by the functional loss of CIC-5 is associated with increased expression of CAIII in both mouse and human PT cells. In order to clarify whether CAIII induction was specifically caused by CIC-5 inactivation or participated in a common cellular response to PT dysfunction, CAIII mRNA expression was investigated in two additional mouse models of renal Fanconi syndrome, namely the megalin- and cystinosin-deficient mice. These models can be distinguished from each other by the severity of PT defects, as summarized in Materials and Methods. The expression of CAIII mRNA was significantly increased in megalin KO (262±22% of WT level, n=3), whereas no changes were observed in Ctns KO samples. Moreover immunoblotting analyses detected a specific excretion of CAIII in the urine of mice lacking megalin (FIG. 6C). These results indicate that the induction of CAIII expression may correlate with the severity of PT dysfunction, suggesting that this isozyme participates in the general cellular response to PT dysfunction.

### 2.7. Induction of CAIII Expression in PT Cells Exposed to H₂O₂

The HK-2 cell line is a well-established model for normal human PT cells (Ryan, M. J., G. Johnson, J. Kirk, S. M. Fuerstenberg, R. A. Zager, B. Torok-Storb. 1994. Kidney Int. 45: 48-57). Exposure of HK-2 cells to H₂O₂ 1 mM induced a significant increase in CAIII mRNA expression as early as 3 hours postincubation, with a maximal level observed at 6 hours posttreatment. Immunoblotting analyses showed an early and stable induction of CAIII from 6 hours postincubation with H₂O₂ (FIG. 10). Similarly, exposure of OK cells to H₂O₂ 0.3 mM was associated with an increased expression of CAIII from 6 hours postincubation (data not shown). These data demonstrate that PT cells rapidly increase their endogeneous expression of CAIII in response to oxidative conditions.

### 2.8. Specificity of CAII and CAIII Antibodies

Extracts from mouse epididymis and human red blood cell ghosts were separated on 14% SDS-PAGE (10×7 cm), blotted on nitrocellulose and probed with anti-CAII or anti-CAIII antibodies as described in the Methods. FIG. 12A shows that in epididymis samples, which express both CAII and CAIII, the anti-CAII antibodies recognize the CAII band at ˜29 kD with minimal cross-reactivity with the CAIII band at ˜27 kD (left panel). By contrast, the anti-CAIII antibodies recognize exclusively the lower band corresponding to CAIII at ˜27 kD (right panel). In FIG. 12B the strong band corresponding to CAII in RBC ghosts (˜29 kD, left lanes) is not detected with anti-CAIII antibodies.

### 2.9. Detection of CAII and CAIII Isozymes in Mouse Urine

Urine samples from Clcn5 wild-type and knock-out mice obtained on protease inhibitors in metabolic cages (see Methods) and normalized for urinary creatinine content were separated on 14% SDS-PAGE (10×7 cm), blotted on nitrocellulose and probed with anti-CAII or anti-CAIII antibodies. In FIG. 13 the strong band shown corresponding to CAII (˜29 kD) is detected with variable intensity in wild-type and knock-out urine samples (upper panel), whereas the band corresponding to CAIII (˜29 kD) is only detected in knock-out samples (lower panel). It is noted that that the film was exposed for 1 h (CAII, 1:2,000 dilution) vs. 5 min (CAIII, 1:7,000 dilution).

### 3. Discussion

Despite the physiological importance of PT cells and the clinical severity of the renal Fanconi syndrome, the cellular and metabolic outcomes of inherited PT dysfunction remain essentially unknown. Using mouse, human and cellular models, we show here that inherited PT dysfunction is associated with higher cell turnover and increased cellular response to oxidant damage, as well as a major upregulation of CAIII, an isozyme that was previously unknown in the kidney. The induction of CAIII in Clcn5^{Y/-} mice was restricted to the kidney, with no changes observed in the other CAIII-expressing organs. These findings were confirmed in a patient harbouring a missense mutation in CLCN5 and another mouse model of PT dysfunction caused by the inactivation of megalin. Moreover the exposure of two distinct PT cell lines to oxidant conditions caused a rapid and consistent response of CAIII.

Due to their intense reabsorptive activity involving active transport, the epithelial cells lining the PT are particularly vulnerable to injury. However, in contrast to brain and heart, the kidney can completely recover from an ischemic or toxic insult. Following cell death by necrosis and apoptosis, the surviving PT cells dedifferentiate and proliferate to eventually replace the injured epithelial cells and restore tubular integrity (Bonventre, J. V. 2003. J. Am. Soc. Nephrol. 14: S55-S61). Our studies reveal that a similar process occurs as a response to a chronic injury, i.e. inherited renal Fanconi syndrome in mouse and man. The proliferative activity of PT cells, assessed using antibodies to cell proliferation-associated nuclear proteins PCNA and KI-67, was almost 4-fold increased, with the involvement of the G1 cell cycle kinase being suggested by the upregulated cyclin E. Furthermore, PT cells underwent dedifferentiation, as indicated by the expression of osteopontin and the mesodermal marker CAIII (see below). These modifications occurred at a time when no visible alterations in PT cell morphology nor changes in renal function were observed (Wang, S. S. 2000. Hum. Mol. Genet. 9: 2937-2945), and without change in the apoptotic rate. The growth and/or transcription factors that could be involved in this adaptative response remain to be defined. In particular, the lack of albumin endocytosis due to impaired receptor-mediated endocytosis in these models, which normally activates various signal transduction cascades in PT cells, may play a major role (Imai, E. et al 2004. Kidney Int. 66: 2085-2087).

An important finding of this study is the demonstration of a significantly increased production of superoxide anion in the Clcn5^{Y/-} PT cells, with the parallel induction of SOD and thioredoxin, pointing to increased oxidative stress and solicitation of cell oxidative defences. Thioredoxin was also selectively increased in a human kidney with inactivating CIC-5 mutation. Increased ROS production, which depletes endogenous radical scavengers and causes cell oxidative damage, is involved in PT lesions induced by nephrotoxic compounds such as cisplatin and heavy metals (Percy, C., B. et al 2005. Adv. Chronic Kidney Dis. 12: 78-83). Similarly, IV iron produces an oxidative stress that is associated with transient proteinuria and tubular damage (Agarwal, R. et al. 2004. Kidney Int. 65: 2279-2289). Furthermore, Wilmer et al. have shown a significantly elevated level of oxidized glutathione in PT cells derived from patients with a renal Fanconi syndrome due to cystinosis (Wilmer, M. J. et al. 2005. Biochem. Biophys. Res. Commun. 337: 610-614). Taken together, these data suggest that acquired and inherited dysfunctions of the PT are associated with a state of oxidative stress. Considering the importance of albumin endocytosis in the regulation of H₂O₂ production in PT cells (Imai, E, et al 2004. Kidney Int. 66: 2085-2087), it is tempting to hypothesize that the lack of albumin endocytosis in PT cells lacking CIC-5 may lead to unbalanced production of H₂0₂, that in turn could contribute to PT dysfunction.

The endocytic defect caused by the loss of CIC-5 is reflected in vivo by a selective depletion of megalin (and its partner cubilin) from the brush border in the absence of morphological lesion. Recently, Guggino et al. suggested that megalin could act as a sensor of albumin and that a decrease in its plasma membrane expression could reduce protein kinase B activity and alter the survival pathway involving phosphorylation of Bad in cultured PT cells. The generalized trafficking defect in PT cells lacking CIC-58 could also impair the translocation/activation of protein kinase B, further reducing defense against cytotoxicity. Furthermore, albumin is known to exert a potent survival activity in mouse PT cells, most likely through scavenging of reactive oxygen species, so that a reduced capacity of albumin uptake may be deleterious. In contrast, excessive albumin endocytosis also promotes H₂O₂ generation in PT cells. Thus, the link between oxidative stress and defective endocytosis remains speculative, and the two events could independently reflect the multiple changes induced by the loss of CIC-5 in PT cells. Type III CA belongs to the family of zinc metallo-enzymes that reversibly hydrate CO₂, thus generating hydrogen and bicarbonate ions essential for acid-base homeostasis, respiration, ureagenesis, lipidogenesis, urinary acidification and bone resorption (Lindskog, S. 1997. Pharmacol. Ther. 74: 1-20; Sly, W. S., P. Y. Hu. 1995. Annu. Rev. Biochem. 64: 375-401). At least 15 different isoforms, with 11 catalytically active isozymes, have been described in the mammals, with distinct kinetic properties and tissue distribution. Subcellularly, four of the active CA isozymes are cytosolic (CAI, CAII, CAIII, and CAVII), four are membrane-bound (CAIV, CAIX, CAXII and CAXIV), two are mitochondrial (CA VA and VB), and one is a secretory isoform (CAVI) (Mori, K. et al. 1999. J. Biol. Chem. 274: 15701-15705). Type II and IV CA represent the two main isozymes in the kidney, located in PT cells where they participate in H⁺ secretion and HCO₃⁻ reabsorption, as well as to NaCl homeostasis. CAII is present in the cytosol of the intercalated cells of the collecting duct, where it ensures net urinary acidification. The functional loss of CAII causes Guibaud-Vainsel disease, an inherited syndrome characterized by renal tubular acidosis, osteopetrosis, and cerebral calcifications (Sly, W. S., et al. 1985. N. Engl. J. Med. 313: 139-145). Two other CA isozymes have been located in mouse kidney, i.e. CAXIII and CAXIV, but their specific role, as well as their interactions with CAII and CAIV in this organ, remain unknown (Lehtonen, J., B. et al, 2004. J. Biol. Chem. 279: 2719-2727., Mori, K., Y. et al. 1999. J. Biol. Chem. 274: 15701-15705).

Type III CA is distinguishable from the other CA isozymes by several features, particularly its resistance to sulfonamide inhibitors and its low CO₂ hydration ability which represents ˜2% of CAII activity (Jewell, D. A. et al., 1991. Biochemistry 30: 1484-1490). Its lower catalytic turnover is in part explained by the replacement of a histidine by a lysine at residue 64, which is not efficient for proton transfer during catalysis. In addition, the phenyl side chain of Phe 198 (instead of Leu in CAII) causes a steric constriction of the CAIII active site, which may also explain the lower catalytic activity and resistance to acetazolamide (Duda, D. M. et al. 2005. Biochemistry. 44: 10046-10053., 25). Although CAIII is abundantly expressed in the cytosol of skeletal muscle cells, adipocytes and hepatocytes, its function and regulation remain unclear (Kim, G., T et al. 2004. Mol. Cell Biol. 24: 9942-9947). In addition, CAIII is an early mesodermal marker expressed in embryonic mouse notochord, that defines a subset of mesodermal cell types later during embryogenesis (Lyons, G. E., et al, Development. 111: 233-244). Along with other genes encoding growth/transcription factors, that recapitulate the expression pattern seen during nephrogenesis (Bonventre, J. V. 2003. J. Am. Soc. Nephroi. 14: S55-S61). CAIII may thus represent a novel marker of cell dedifferentation associated with inherited PT dysfunction.

CAIII is known to function in an oxidizing environment (Cabiscol, E., R. L. Levine. 1995. J. Biol. Chem. 270: 14742-14747), which is of interest when considering the oxidative stress evidenced in the mouse and human PT cells investigated here. Two reactive sulfhydryl groups of CAIII are rapidly S-thiolated by glutathione after in vivo and in vitro exposure to oxidative conditions, and dethiolated by enzymatic reactions (glutaredoxin and thioredoxin-like) (Chai, Y. C et al. 1991. Arch. Biochem. Biophys. 284: 270-288). CAIII also plays a protective role against H₂O₂-induced apoptosis, contrasting with the lack of effect of the CAII isoform (Raisanen, S. R., et al 1999. FASEB J. 13: 513-522). Furthermore, NIH/3T3 cells overexpressing CAIII grow faster and are more resistant to cytotoxic concentrations of H₂O₂ than control cells. Thus, by analogy with acute (ischemia-reperfusion) and chronic (aging) injuries (Cabiscol, E., R. L. Levine. 1995. J. Biol. Chem. 270: 14742-14747., Eaton, P., et al. 2003. J. Am. Soc. Nephrol. 14: S290-S296), CAIII may function as an oxyradical scavenger, protecting PT cells from the oxidative stress induced by chronic dysfunction. Of note, Kim et al. have postulated that CAIII may have evolved into a percarbonic acid anhydrase, which would mediate H₂O₂+CO₂ [symbol] H₂CO₄ (Richardson, D. E., et al. 2003. Free Radic. Biol. Med. 35: 1538-1550, and Kim, G et al. 2004. Mol. Cell Biol. 24: 9942-9947).

What could be the regulator of CAIII induction associated with PT dysfunction? The concentration of CAIII in rat male liver was found to be 30 times greater than that in females, with a marked reduction after castration, suggesting regulation by androgens (Carter, N., et al 2001. Ups J. Med. Sci. 106: 67-76). However, comparative real-time RT-PCR studies in mouse kidney did not find any significant difference in CAIII mRNA expression between 15-week-old males and females (n=5, data not shown). Thyroidectomy is known to increase CAIII concentration in rat muscle (Jeffery, S., J. Histochem. Cytochem. 35: 663-668), which could be relevant when considering that CIC-5 is highly expressed in wild-type mouse thyroid (van den Hove, M.-F. et al 2006. Endocrinology. 147: 1287-1296). However, mice lacking CIC-5 develop a euthyoid goiter, without alterations in circulating T4 and TSH levels. In contrast, preliminary data suggest that the transcription factor hepatocyte nuclear factor 1alpha (HNF1α), which is expressed in liver, pancreas, intestine and kidney, may regulate the transcription of CAIII in PT cells. In silico analysis of the promoter of the Car3 gene revealed several potential HNF1 binding sites, and mice lacking HNF1α, which show a severe PT dysfunction reflected by polyuria, glucosuria, aminoaciduria and phosphaturia, are characterized by a decreased renal expression of CAIII (F. Jouret, K. Parreira and O. Devuyst, unpublished observations).

Finally, the selective detection of CAIII in the urine of patients and mice lacking CIC-5 and other congenital and acquired models of PT dysfunction (F. Jouret and O. Devuyst, unpublished data) suggests that the urinary excretion of CAIII may be a useful biomarker of renal Fanconi syndrome. As compared with traditional markers, such as low-molecular-weight proteins (e.g. β2-microglobulin), which are produced outside the kidney, filtered by the glomerulus but not reabsorbed due to defective PT endocytosis, or PT cell components (such as N-acetyl-beta-glucosaminidase, NAG) that appear in the urine in case of structural alterations, CAIII in the urine may directly reflect a state of cellular dysfunction, without changes in renal function or morphology. Furthermore, the upregulation of CAIII is organ- and segment-specific, and clearly conserved in mouse and man.

Considering that CAIII is an early mesodermal marker, it may reflect cell dedifferentiation along with other genes encoding growth and transcription factors that recapitulate the expression pattern seen during nephrogenesis.

In conclusion, we report on CAIII, a novel kidney CA isozyme that is specifically induced in scattered mouse and human PT cells, where it may participate to the cellular response against oxidative damage associated with chronic PT dysfunction.

### 4. Enzymatic Assay of Carbonic Anhydrase (Ec 4.2.1.1) Tris-Sulfate Buffer

### Principle:

p-Nitrophenyl Acetate+H₂O→p-Nitrophenol+Acetate Carbonic Anhydrase

CONDITIONS: T=0° C., pH=7.6, A348 nm, Light path=1 cm

### METHOD: Continuous Spectrophotometric Rate Determination

### Reagents:

A. 15 mM Tris Sulfate Buffer, pH 7.6 at 0° C. (Prepare 100 ml in deionized water using Trizma Sulfate, Sigma product No T-8379. Adjust to pH 7.6 at 0° C. with 1 M NaOH.)
B. 3 mM p-Nitrophenyl Acetate Solution (PNPA) (Prepare 25 ml in deionized water using p-Nitrophenyl Acetate, Sigma product No 8130. Facilitate by first dissolving 13.6 mg of p-Nitrophenyl Acetate in 1 ml of Acetone. Then raise the volume to 25 ml with deionized water. PREPARE FRESH.
C. Carbonic Anhydrase Enzyme Solution (Immediately before use, prepare a solution containing 100-200 units/ml of Carbonic Anhydrase in Reagent A.)

### Procedure:

Pipette (in milliliters) the following reagents into suitable cuvettes:

| | Test | Blank |
|---|---|---|
| Reagent A (Buffer) | 1.90 | 2.00 |
| Reagent B (PNPA) | 1.00 | 1.00 |

Mix by inversion and equilibrate to 0° C. Then add:

| | Test | Blank |
|---|---|---|
| Reagent C (Enzyme Solution) | 0.10 | — |

Immediately mix by inversion and record the increase in A348 nm for approximately 5 minutes. Obtain the r A348 nm/minute using the maximum linear rate for both the Test and Blank.

### Calculations:

Units/mg enzyme is calculated as:

[(ΔA348 nm/min Test−ΔA348 nm/min Blank)(1000)]/[(5.0)(mg enzyme/ml RM)]

with:

1000=Conversion to micromoles

5=Millimolar extinction coefficient of p-Nitrophenol at pH

7.6 at 0° C.

RM=Reaction Mix

### Unit Definition:

Multiply Units/mg by 1.5 to obtain Sigma Units.

Sigma Unit Definition: One Wilbur-Anderson (W-A) unit will cause the pH of a 0.012 M Veronal buffer to drop from 8.3 to 6.3 per minute at 0 C. (One W-A unit is essentially equivalent to one Roughton-Booth unit.)

### Final Assay Concentration:

In a 3 ml reaction mix, the final concentrations are 10 mM Tris sulfate, 1 mM p-nitrophenyl acetate and 10-20 units carbonic anhydrase.

1. A method for determining a condition related to renal proximal tubule (RPT) dysfunction in a subject comprising measuring the presence of type III Carbonic Anhydrase, CAIII, in a urine sample of said subject and determining said condition when said measured presence is different from the measured presence in a urine sample of a healthy subject.

2. Method according to claim 1, wherein measuring said presence is performed by measuring the concentration of CAIII in said samples.

3. Method according to claim 1, wherein measuring said presence is performed by measuring CAIII enzyme activity in said samples.

4. Method according to claim 1, comprising the steps of:
(i) obtaining a urine sample from a subject;
(ii) measuring the concentration of CAIII in the sample;
(iii) comparing the concentration of CAIII in the sample with the concentration of CAIII in a healthy subject; and
(iv) determining a condition related to dysfunction of the RPT when the concentration of CAIII in the sample is different, and preferably at least 10% different, from that measured in a sample from a healthy subject.

5. Method according to claim 1, comprising the steps of:
(i) obtaining a urine sample from a subject;
(ii) measuring the concentration of CAIII in the sample;
(iii) determining a condition related to dysfunction of the RPT when detectable CAIII is present in the sample or concentration of CAIII in the sample is greater than or equal to a threshold concentration.

6. Method according to claim 5 wherein said threshold value is between 1 pM and 1 mM.

7. Method according to claim 1 comprising the steps of:
(i) obtaining a urine sample from a subject;
(ii) measuring the CAIII enzyme activity in the sample;
(iii) comparing the CAIII enzyme activity in the sample with the CAIII enzyme activity in a healthy subject; and
(iv) determining a condition related to dysfunction of the RPT when the CAIII enzyme activity in the sample is different, and preferably at least 10% different, from that measured in a sample from a healthy subject.

8. Method according to claim 1 comprising the steps of:
(i) obtaining a urine sample from a subject;
(ii) measuring the CAIII enzyme activity in the sample;
(iii) determining a condition related to dysfunction of the RPT when the CAIII enzyme activity in the sample is greater than or equal to a threshold enzyme activity.

9. Method for monitoring the progress of a condition related to renal proximal tubule (RPT) dysfunction in a subject by monitoring the presence of CAIII in two or more urine samples taken at different intervals.

10. Method according to claim 9, comprising the steps of:
(i) obtaining two or more urine samples from a subject, taken at different time intervals;
(ii) measuring the concentration of CAIII in each sample;
(iii) determining the progress of a condition related to dysfunction of the RPT by comparing the concentrations of CAIII in the measured samples over time.

11. Method according to claim 9, comprising the steps of:
(i) obtaining two or more urine samples from a subject, taken at different time intervals;
(ii) measuring the CAIII enzyme activity in each sample;
(iii) determining the progress of a condition related to dysfunction of the RPT by comparing the CAIII enzyme activities in the measured samples over time.

12. Method for monitoring the efficacy of a treatment of a condition related to renal proximal tubule (RPT) dysfunction in a subject by measuring the presence of CAIII in a urine sample taken before, after and optionally during treatment.

13. Method according to claim 12, comprising the steps of:
(i) obtaining a pre-administration urine sample from a subject prior to administration of the treatment;
(ii) measuring the presence of CAIII in the pre-administration sample;
(iii) obtaining one or more post-administration urine samples from the subject;
(iv) measuring the presence of CAIII the post-administration samples;
(v) comparing the presence of CAIII in the pre-administration sample with the presence of CAIII in the post-administration sample or samples; and
(vi) determining the efficacy of a treatment.

14. Method according to claim 12, wherein measuring said presence is performed by measuring the concentration of CAIII in said pre-administration and post-administration samples.

15. Method according to claim 12, wherein measuring said presence is performed by measuring CAIII enzyme activity in said pre-administration and post-administration samples.

16. Method according to claim 12, further comprising the step of altering the treatment to decrease the concentration or enzyme activity of CAIII in said post-administration samples.

17. Method according to claim 1, 9 or 12, wherein the concentration of CAIII in a sample is measured by using a CAIII specific probe.

18. Method according to claim 17, wherein said CAM probe is an antibody directed against CAM or a fragment thereof.

19. Method according to claim 18 wherein said antibody is a polyclonal antibody, monoclonal antibody, humanised or chimeric antibody, engineered antibody, or biologically functional antibody fragments sufficient for binding to CAIII.

20. Method according to claim 19, wherein said antibody is mouse monoclonal antibody clone 2CA-4.

21. Method according to claim 1, 9 or 12, wherein said CAIII is human CAM, a polypeptide having the sequence represented by SEQ ID NO: 1, or a fragment thereof.

22. Method according to claim 17, wherein the concentration of CAIII is measured using any of biochemical assay, immunoassay, surface plasmon resonance, fluorescence resonance energy transfer, bioluminescence resonance energy transfer or quenching.

23. Method according to claim 1, wherein said condition is inherited or acquired.

24. Method according to claim 1, wherein said inherited condition related to renal proximal tubule (RPT) dysfunction is selected form the group comprising COX deficiency, Cystinosis, Dent's disease (1), Dent's disease (2), Fanconi-Bickel syndrome, Fructosaemia, Galactosaemia, Imerslund-Grasbeck disease, Lowe syndrome, Tyrosinaemia, von Gierke disease, and Wilson disease.

25. Method according to claim 1, wherein said acquired condition related to renal proximal tubule (RPT) dysfunction is selected from the group comprising nephrotoxicity, renal injury, acute or chronic renal or kidney failure, multiple myeloma, light chain deposition disease and renal transplantation.

26. Method according to claim 1, further comprising detecting the presence of proteins and sugar in the urine.

27. Method for diagnosing a condition related to renal proximal tubule (RPT) dysfunction in a subject comprising measuring the presence of type III Carbonic Anhydrase, CAIII, in a urine sample of said subject.

28. Method according to claim 27 for preventive screening of subjects for the presence of a condition related to renal proximal tubule (RPT) dysfunction in said subject.

29. Method according to claim 27 for monitoring kidney transplantation in a subject.

30. Device for determining a condition related to dysfunction of the RPT in a subject, wherein said device comprises at least one CAIII specific probe as defined in claim 17 for measuring the presence of CAIII in an urine sample, and a solid support whereby said CAIII is immobilised thereon, wherein said solid support has fluid capillary properties and comprises:
a distal and proximal end,
a sample application zone in the vicinity of the proximal end,
a reaction zone distal to the sample application zone,
a detection zone distal to the reaction zone,
where the reaction zone is disposed with CAIII probe labelled with detection agent, that can migrate towards the distal end in a flow of fluid by capillary action,
where the detection zone comprises said immobilised CAIII probe that can capture CAM.

31. Device according to claim 30 housed in a cartridge watertight against urine, having an opening to provide access to the application zone in proximal end, and another opening to enable reading of detection zone close to the distal end.

32. Device according to claim 31, wherein said cartridge is disposed with a sensor code for communicating with a reading device.

33. Device for determining a condition related to renal proximal tubule (RPT) dysfunction in a subject comprising a reagent strip wherein said strip comprises a solid support provided with at least one test pad for measuring the presence of CAIII in an urine sample.

34. Device according to claim 33 wherein said test pad comprises a carrier matrix incorporating a reagent composition capable of interacting with CAIII to produce a measurable response.

35. Device according to claim 33 wherein said solid support further comprises one or more test pads for measuring the presence of one or more analytes selected from the group comprising proteins, blood, leukocytes, nitrite, glucose, ketones, creatinine, albumin, bilirubin, urobilinogen, and/or pH test pad and/or a test pad for measuring specific gravity.

36. (canceled)

37. Test pad comprising a carrier matrix incorporating a reagent composition capable of interacting with CAIII to produce a measurable response.

38. (canceled)

39. Kit comprising a device according to claim 30 and a urine sample container and/or control standards comprising CAIII.


SEQUENCE LISTING (48 sequences)

SEQ ID NO: 1 - protein, 260 aa, Homo sapiens
MAKEWGYASHNGPDHWHELFPNAKGENQSPVELHTKDIRHDPSLQPWSVSYDGGSAKTILNNGKTCRVVFDDTYDRSMLRGGPLPGPYRLRQFHLHWGSSDDHGSEHTVDGVKYAAELHLVHWNPKYNTFKEALKQRDGIAVIGIFLKIGHENGEFQIFLDALDKIKTKGKEAPFTKFDPSCLFPACRDYWTYQGSFTTPPCEECIVWLLLKEPMTVSSDQMAKLRSLLSSAENEPPVPLVSNWRPPQPINNRVVRASFK

SEQ ID NO: 2 - DNA, 17 bp, Artificial sequence
  AFLP primer/adapter
ctcgtagactgcgtacc

SEQ ID NO: 3 - DNA, 18 bp, Artificial sequence
  AFLP primer/adapter
aattggtacgcagtctac

SEQ ID NO: 4 - DNA, 16 bp, Artificial sequence
  AFLP primer/adapter
gacgatgagtcctgag

SEQ ID NO: 5 - DNA, 14 bp, Artificial sequence
  AFLP primer/adapter
tactcaggactcat

SEQ ID NO: 6 - DNA, 16 bp, Artificial sequence
  AFLP primer/adapter
gactgcgtaccaattc

SEQ ID NO: 7 - DNA, 16 bp, Artificial sequence
  AFLP primer/adapter
gatgagtcctgagtaa

SEQ ID NO: 8 - DNA, 18 bp, Artificial sequence
  AFLP primer/adapter
gactgcgtaccaattcaa

SEQ ID NO: 9 - DNA, 18 bp, Artificial sequence
  AFLP primer/adapter
gactgcgtaccaattcac

SEQ ID NO: 10 - DNA, 18 bp, Artificial sequence
  AFLP primer/adapter
gactgcgtaccaattcag

SEQ ID NO: 11 - DNA, 18 bp, Artificial sequence
  AFLP primer/adapter
gactgcgtaccaattcat

SEQ ID NO: 12 - DNA, 18 bp, Artificial sequence
  AFLP primer/adapter
gatgagtcctgagtaaaa

SEQ ID NO: 13 - DNA, 18 bp, Artificial sequence
  AFLP primer/adapter
gatgagtcctgagtaaac

SEQ ID NO: 14 - DNA, 18 bp, Artificial sequence
  AFLP primer/adapter
gatgagtcctgagtaaat

SEQ ID NO: 15 - DNA, 20 bp, Artificial sequence
  PCR primer
cttgaagcactgcattccat

SEQ ID NO: 16 - DNA, 20 bp, Artificial sequence
  PCR primer
cttgatgccctggacaaaat

SEQ ID NO: 17 - DNA, 20 bp, Artificial sequence
  PCR primer
ttggaatcccagaacaggag

SEQ ID NO: 18 - DNA, 20 bp, Artificial sequence
  PCR primer
tgcaaaggtagaggctccat

SEQ ID NO: 19 - DNA, 20 bp, Artificial sequence
  PCR primer
tccaatcgtccctacagtcg

SEQ ID NO: 20 - DNA, 20 bp, Artificial sequence
  PCR primer
catggtctgggacttctgga

SEQ ID NO: 21 - DNA, 20 bp, Artificial sequence
  PCR primer
gggttccacgtccatcagta

SEQ ID NO: 22 - DNA, 20 bp, Artificial sequence
  PCR primer
tgatcaagcccttcttccat

SEQ ID NO: 23 - DNA, 20 bp, Artificial sequence
  PCR primer
tgcaccaccaactgcttagc

SEQ ID NO: 24 - DNA, 20 bp, Artificial sequence
  PCR primer
acattgtggccctctgtgtg

SEQ ID NO: 25 - DNA, 20 bp, Artificial sequence
  PCR primer
cacgatccaggtcacacatt

SEQ ID NO: 26 - DNA, 20 bp, Artificial sequence
  PCR primer
gagccgtggtaggtccaata

SEQ ID NO: 27 - DNA, 20 bp, Artificial sequence
  PCR primer
attgccaagctctccacttg

SEQ ID NO: 28 - DNA, 19 bp, Artificial sequence
  PCR primer
caggtaggccagagcaagt

SEQ ID NO: 29 - DNA, 20 bp, Artificial sequence
  PCR primer
cgctcttcatgtgagaggtg

SEQ ID NO: 30 - DNA, 20 bp, Artificial sequence
  PCR primer
gactgcctctccatctgcat

SEQ ID NO: 31 - DNA, 20 bp, Artificial sequence
  PCR primer
cagtcacattgcccaggtct

SEQ ID NO: 32 - DNA, 20 bp, Artificial sequence
  PCR primer
cccaccttttgacccttttt

SEQ ID NO: 33 - DNA, 20 bp, Artificial sequence
  PCR primer
ggatgcagggatgatgttct

SEQ ID NO: 34 - DNA, 20 bp, Artificial sequence
  PCR primer
ttatgtcccccgttgactga

SEQ ID NO: 35 - DNA, 19 bp, Artificial sequence
  PCR primer
ccctggatggcacttacag

SEQ ID NO: 36 - DNA, 19 bp, Artificial sequence
  PCR primer
gccgggactactggaccta

SEQ ID NO: 37 - DNA, 20 bp, Artificial sequence
  PCR primer
acgtctctttggtgcagctc

SEQ ID NO: 38 - DNA, 20 bp, Artificial sequence
  PCR primer
atggccgaggtgatagtgtg

SEQ ID NO: 39 - DNA, 20 bp, Artificial sequence
  PCR primer
tggctcattttgaccgagag

SEQ ID NO: 40 - DNA, 18 bp, Artificial sequence
  PCR primer
tcagccacgtggtgtggg

SEQ ID NO: 41 - DNA, 20 bp, Artificial sequence
  PCR primer
ggggctctccagaacatcat

SEQ ID NO: 42 - DNA, 24 bp, Artificial sequence
  PCR primer
cagctttcccaaaatccccacatt

SEQ ID NO: 43 - DNA, 19 bp, Artificial sequence
  PCR primer
cgttctcagcactggagag

SEQ ID NO: 44 - DNA, 20 bp, Artificial sequence
  PCR primer
gcgttatcttcggcccttag

SEQ ID NO: 45 - DNA, 20 bp, Artificial sequence
  PCR primer
gatggccttgtatgcaccat

SEQ ID NO: 46 - DNA, 20 bp, Artificial sequence
  PCR primer
gcgatgggagtcttctttcc

SEQ ID NO: 47 - DNA, 22 bp, Artificial sequence
  PCR primer
tggaatgttggcatgcatttga

SEQ ID NO: 48 - DNA, 19 bp, Artificial sequence
  PCR primer
tctagacggcaggtcaggt